SEQUENCE LISTING (5 sequences)

SEQ ID NO: 1 - protein, 17 aa, Artificial Sequence
  Description of Artificial Sequence Synthetic peptide
APLPRWSGPIGVSWGLR

SEQ ID NO: 2 - protein, 44 aa, Artificial Sequence
  Description of Artificial Sequence Synthetic polypeptide
QDRLDAPPPPAAPLPRWSGPIGVSWGLRAAAAGGAFPRGGRWRR

SEQ ID NO: 3 - protein, 12 aa, Artificial Sequence
  Description of Artificial Sequence Synthetic peptide
WSGPIGVSWGLR

SEQ ID NO: 4 - protein, 5 aa, Artificial Sequence
  Description of Artificial Sequence Synthetic peptide
APLPR

SEQ ID NO: 5 - protein, 16 aa, Artificial Sequence
  Description of Artificial Sequence Synthetic peptide
QDRLDAPPPPAAPLPR

(19) United States
(12) Patent Application Publication
(10) Pub. No.: US 2012/0322060 A1
(43) Pub. Date: Dec. 20, 2012

(54) DIAGNOSIS AND MONITORING TREATMENT OF PSYCHIATRIC DISEASES WITH SPADIN AND RELATED METHODS

(75) Inventors: JEAN MAZELLA, BIOT (FR); MARC BORSOTTO, GRASSE (FR); CATHERINE HEURTEAUX, LA COLLE SUR LOUP (FR); CÉCILE LE SAINT, QUEBEC (CA); ERIC CHEVET, BORDEAUX (FR); ANNY CUPO, JUAN-LES-PINS (FR); SAID TAOUJI, VILLENAVE D'ORNON (FR)

(73) Assignee: CENTRE NATIONAL DE LE RECHERCHE SCIENTIFIQUE - CNRS -, PARIS CEDEX (FR)

(21) Appl. No.: 13/441,563

(22) Filed: Apr. 6, 2012

Related U.S. Application Data
(60) Provisional application No. 61/475,085, filed on Apr. 13, 2011.

(51) Int. Cl. G01N 33/566; C07K 16/18; C07H 21/04; C12Q 1/68; G01N 27/26

(52) U.S. Cl. 435/6.11; 435/6.12; 435/7.21; 436/501; 536/24.31; 530/391.3

(57) ABSTRACT

A method of diagnosing depression in a subject, comprising analyzing a biological sample from a subject in need of diagnosis of depression for the expression of spadin, detecting and measuring the amount of spadin in the sample, and diagnosing depression in the subject if an increase or a decrease in spadin expression in the biological sample is detected compared to a control, is described as are methods for monitoring treatment, remission and the course of the disease. Related kits are also described. Also described is a method for identifying candidate compounds for treating depression.

### CROSS REFERENCE TO RELATED APPLICATION

This application claims priority of U.S. Provisional Application No. 61/475,085, filed on Apr. 13, 2011, the disclosure of which is hereby incorporated in its entirety by reference.

### REFERENCE TO SEQUENCE LISTING

A paper copy of a PatentIn version 3.5 sequence listing identifying SEQ ID NO: 1-4 is attached and incorporated into the specification by reference.

### TECHNICAL DOMAIN OF THE INVENTION

The present invention relates to spadin, a secreted propeptide derived from the propeptide generated by the maturation of the neurotensin receptor 3 (NTSR3/Sortilin) and acting through TREK-1 inhibition, and its use in the diagnosis of psychiatric diseases. The present invention particularly relates to the use of this peptide as a tool diagnostic of depression.

The present invention finds application in the pharmaceutical industry and in particular in the development of diagnostic tools used in the prevention and/or monitoring of psychiatric diseases, in particular of patients suffering or having suffered from depression.

In the specification below, references in brackets ([ ]) refer to the list of references at the end of the examples.

### PRIOR ART

Psychiatric diseases are a real Public Health problem. The most recent studies have confirmed the high prevalence of depression: in their lifetime, 20% of women and 10% of men had, have or will have a depressive episode as described in [59]. Such statistics are clearly significant; especially when looking at the major complication of depression, suicide, which stood at 12,000 deaths per year in countries like France as described in [60].

Depression is a disease very common and often disabling. It can affect up to 20% of the population in industrialized countries. Its origins are many and varied. This disease affects both the mind and the behavior and physiology of patients. Treatments for depression are also multiple and searching novel effective treatments for this destructive disease is challenging, in part because of the inherent complexity of the disease itself, but also because there is a lack of knowledge concerning the mechanisms of action of antidepressants (ADs). The World Health Organization (WHO) states that unipolar depression will be the second cause of disability in 2020.

Current antidepressant treatments are inadequate for many individuals (one third of patients are resistant to existing treatments) and, when they are effective, they require several weeks of administration before a therapeutic effect can be observed. However if the antidepressants improve patients' conditions in about 70% of the cases, they only do a complete remission of the disease in 30 to 40% of them. Improving the treatment and/or management of depression is challenging.

Recently, the two-pore domain potassium channel TREK-1 has been identified as a new target in depression. In mice, deletion of TREK-1 gene results in a depression-resistant phenotype that mimics antidepressant treatments. Clearly, the recent Sequenced Treatment Alternatives to Relieve Depression (STAR*D) study [4], a multicenter, prospective, effectiveness trial in major depressive disorder (MDD) has pharmacogenetically identified TREK-1 as a gene implicated in AD response in humans. This study also suggests the utility of animal models for the research of AD treatments in identifying candidate genes for study in humans. Thanks to these animal models we have identified spadin as a potential new concept in AD drug design. Specific blockers of TREK-1 channels (such as spadin, called propeptide in the International Application WO2009/103898 [58], which is incorporated herein in its entirety by reference) may represent a new concept in the field of depression drug design.

Recently, mouse models of depression have highlighted the putative role of the TREK-1 channel in the mechanisms of action of antidepressants. Deletion of the TREK-1 gene (also called kcnk2) results in a depression-resistant phenotype that mimics treatment with antidepressants [1]. TREK-1-deficient mice (kcnk2^{-/-}) display an increased efficiency of 5-HT neurotransmission, a blunted corticosterone response to stress and an increased neurogenesis induced by selective serotonin reuptake inhibitors (SSRIs). The involvement of the TREK-1 protein in mood regulation may be related to its two following specific properties: TREK-1 (i) is directly inhibited by SSRIs [1] and by activated protein kinases A and C, (ii) is potentially linked to G-protein—coupled receptors like the 5-HT_{1A} receptor [2, 3], suggesting that this channel may participate in a 5-HT_{1A} receptor-dependent negative feedback loop. More interestingly, the Star*D study has identified an association between the existence of four genetic variants (SNPs) in the TREK-1 locus, and resistance to multiple antidepressant classes [4]. All these findings indicate that 1) genetic variations in TREK-1 may identify individuals at risk for depression treatment resistance and 2) a search of selective blockers of TREK-1, hitherto not available, might potentially lead to a new generation of antidepressants.

Growing evidence indicates that trafficking and addressing as well as functional properties of native ion channels depend on their lipidic and proteic environments. K⁺ channels are known to interact with partner proteins that are crucial for their regulation. To date, the only identified partner proteins of TREK-1 channels are the A-kinase anchoring protein AKAP150 [5] and the microtubule-associated protein Mtap2 [6] that enhance TREK-1 channel surface expression and current densities. As a consequence of both, its role in the sorting of membrane proteins, and of a cerebral localization similar to that of TREK-1 [7,8], we investigated the possible role of the neurotensin (NT) receptor 3 (NTSR3, also called gp95/sortilin) [9] in the regulation of the channel function. NTSR3/Sortilin is a 95-100-kDa type-1 membrane protein, consisting of a large luminal domain, a single transmembrane segment and a short C-terminal cytoplasmic tail. A large part of NTSR3/Sortilin is localized at the level of the Golgi apparatus where the protein triggers intracellular functions of trafficking. Indeed, the C-terminus of NTSR3/Sortilin interacts with the VHS domain of the sorting protein GGA2 (Golgi-localizing, g-adaptin ear homology domain, ADP-ribosylation factor-binding protein) [10]. This interaction confers to NTSR3/Sortilin the property to sort SAP (sphingolipid activator proteins) to lyzosomes [11]. Depending on its cellular location, NTSR3/Sortilin may also act as a receptor or a co-receptor and binds neurotensin, the precursor of the Nerve Growth Factor (proNGF), the receptor-associated protein (RAP), the lipoprotein lipase and the propeptide released from its precursor form. For example, this receptor is essential to proNGF induction of neuronal cell death via a complex formed with the p75^{NTR} within the cell membrane [12]. In the rat brain, NTSR3/Sortilin as well as TREK-1 are highly expressed in cerebral structures involved in the pathophysiology of depression [13], such as prefrontal and cingulate cortice, amygdala, hippocampus, nucleus accumbens, dorsal raphe and hypothalamus [7, 8]. NTSR3/Sortilin is synthesized as a proform (prosortilin) which, in late Golgi compartments, is converted to the functional ligand-binding receptor by cleavage and release of a 44 residue N-terminal propeptide (Gln¹-Arg⁴⁴, propeptide) by furin [14]. Propeptide binds to the mature receptor with a high affinity (Kd ˜5 nM). Structure-function relationship studies have identified that the peptide Gln¹-Arg²⁸ was as efficient on the binding activity as the entire propeptide Gln¹-Arg⁴⁴, whereas the affinity of the peptide Gln¹-Arg¹⁶ was very low [15].

To our knowledge a marker for the depression is not available.

Therefore finding a depression marker would be of a crucial importance in term of Public Health to prevent, diagnose and/or monitor patients suffering or likely to suffer from psychiatric diseases, in particular from depression.

### SUMMARY OF THE INVENTION

In one embodiment, the invention is directed to a method of diagnosing a psychiatric disease, such as depression in a subject, comprising analyzing a biological sample from a subject in need of diagnosis of the psychiatric disease for the expression of spadin, detecting and measuring the amount of spadin in the sample, and diagnosing psychiatric disease in the subject if an increase in spadin expression in the biological sample is detected compared to a control.

In another embodiment, the invention is directed to a method of diagnosing psychiatric disease, such a depression, in a subject, comprising analyzing a biological sample from a subject in need of diagnosis of psychiatric disease for the expression of spadin, detecting and measuring the amount of spadin in the sample, and diagnosing psychiatric disease in the subject if a decrease in spadin expression in the biological sample is detected compared to a control.

The biological sample is a body fluid used in the above embodiments may be selected from the group consisting of blood, blood plasma, serum, bone marrow, stool, synovial fluid, lymphatic fluid, cerebrospinal fluid, sputum, urine, mother's milk, sperm, exudates and mixtures thereof.

The control, according to the invention, may be the amount of spadin in a sample from a subject without a psychiatric disease, such as depression or the amount of spadin in a sample from a subject with a psychiatric disease, such as depression.

In one embodiment, the analyzing of spadin expression may comprise using a probe, such as an antibody that specifically binds spadin. In a preferred embodiment, such antibody binds SEQ ID NO: 1. In another embodiment, the invention is a nucleic acid probe that binds spadin-encoding nucleic acid. In another embodiment, the analyzing of the biological sample for spadin expression comprises use of nucleic acid amplification.

The above diagnostic methods may be used in combination with other methods of diagnosing psychiatric diseases.

In another embodiment, the invention is directed to a method of monitoring subjects being treated for psychiatric disease, such as depression, comprising: a) detecting and measuring the amount of spadin in a biological sample from a subject being treated for psychiatric disease and b) comparing the amount in a) to the amount of spadin in a control, wherein a change in the amount of spadin is an indication that the treatment is effective.

In another embodiment, the invention is directed to a method of monitoring psychiatric diseased subjects in remission, comprising: a) detecting and measuring the amount of spadin in a biological sample from a subject thought to be in remission for psychiatric disease and b) comparing the amount in a) to the amount of spadin in a healthy control, wherein when the amount in a) is similar to the amount in the healthy control, there is an indication that the subject is in remission.

In another embodiment, the invention is directed to a method of determining the course of psychiatric disease, such as depression, in a subject comprising: a) measuring at a first time, the amount of spadin in a biological sample from the subject; b) measuring, at a second time, the amount of spadin in a biological sample from the subject; and c) comparing the first measurement with the second measurement; wherein the comparative measurements determine the course of the psychiatric disease in the subject.

In another embodiment, the invention is directed to a method of identifying a candidate compound for treating depression, the method comprising determining whether the candidate compound inhibits currents mediated by the TREK-1 channel, wherein when the candidate compound inhibits currents mediated by the TREK-1 channel, it is a candidate compound for treating depression.

In another embodiment, the invention is directed to a kit for carrying out the methods of the invention, the kit comprising a detectably labeled probe for detecting spadin in a biological sample.

### BRIEF DESCRIPTION OF THE DRAWINGS

FIG. 1. NTSR3/Sortilin and Spadin interact with the TREK-1 channel

A, Immunoprecipitation of NTSR3/Sortilin with anti-TREK-1 antibodies (IP α-TREK-1) or of TREK-1 with anti-NTSR3/Sortilin antibodies (IP α-Sort) from transfected COS-7 cells or mouse cortical neurons. Immunoprecipitated proteins were subjected to Western blots and revealed using anti-sortilin (WB: α-Sort) or anti-TREK-1 (WB: α-TREK-1). B, Double immunofluorescence labeling of TREK-1 (Green) and NTSR3/Sortilin (Red) in mouse cortical neurons. Nuclei were labeled using Dapi (Blue) and co-localized proteins were visualized using merge images (arrows), scale bar, 10 μm. C, Influence of NTSR3/Sortilin on the expression of TREK-1 at the plasma membranes. COS-7 cells were transfected with TREK-1 in the absence or in the presence of NTSR3/Sortilin. Crude homogenates, purified plasma membrane proteins or cell surface biotinylated proteins were subjected to Western blot analysis and revealed using anti-TREK-1 antibodies. D, Competition between ¹²⁵I-NT and unlabeled Spadin (closed circles) or NT (open circles) for binding to C13NJ cell homogenates. Each point represents the mean of duplicate determinations from 3 independent experiments. E, Competition between ¹²⁵I-Spadin and unlabeled Spadin (closed circles), NT (open circles) or N-terminal fragment Gln1-Arg 16 (Nterm1-16, open triangles) for binding to TREK-1 transfected COS-7 cell homogenates. Each point represents the mean of duplicate determinations from 2 to 5 independent experiments. Note that non-transfected COS-7 cells were totally devoid of ¹²⁵I-spadin binding. F, Association kinetics of ¹²⁵I-Spadin binding to COS-7 cells transfected with TREK-1. At the indicated times, cells were either washed twice with 500 of binding buffer (closed circles) or treated with 500 μl of acid-NaCl buffer for 2 min (open circles).

FIG. 2. AlphaScreen™ Assays

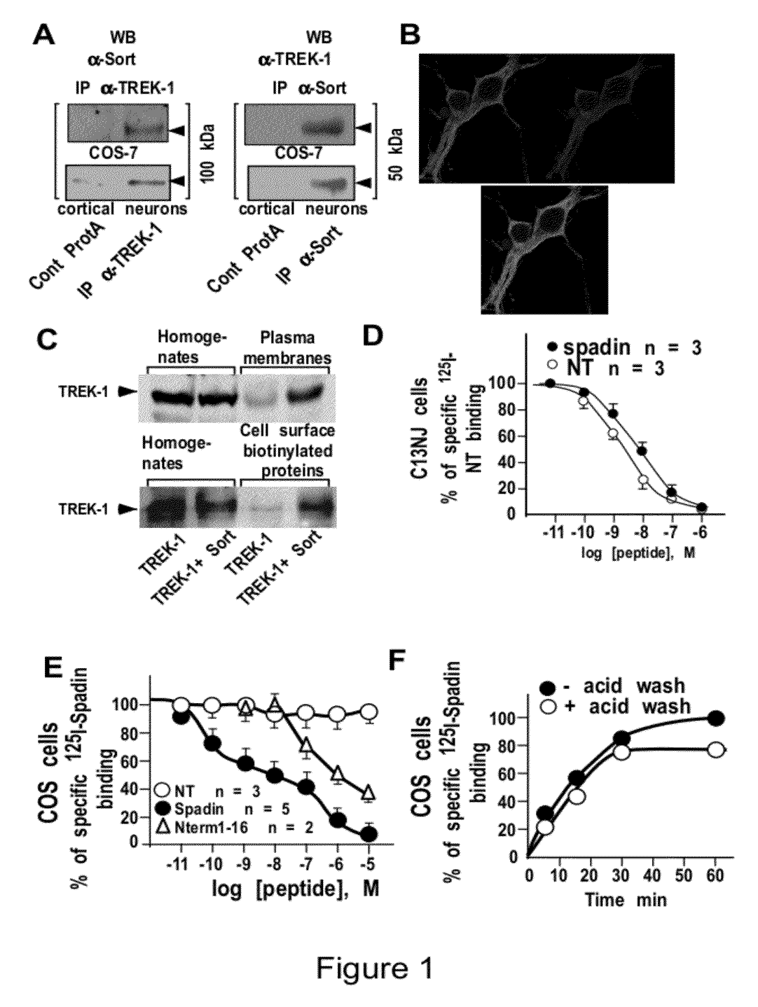

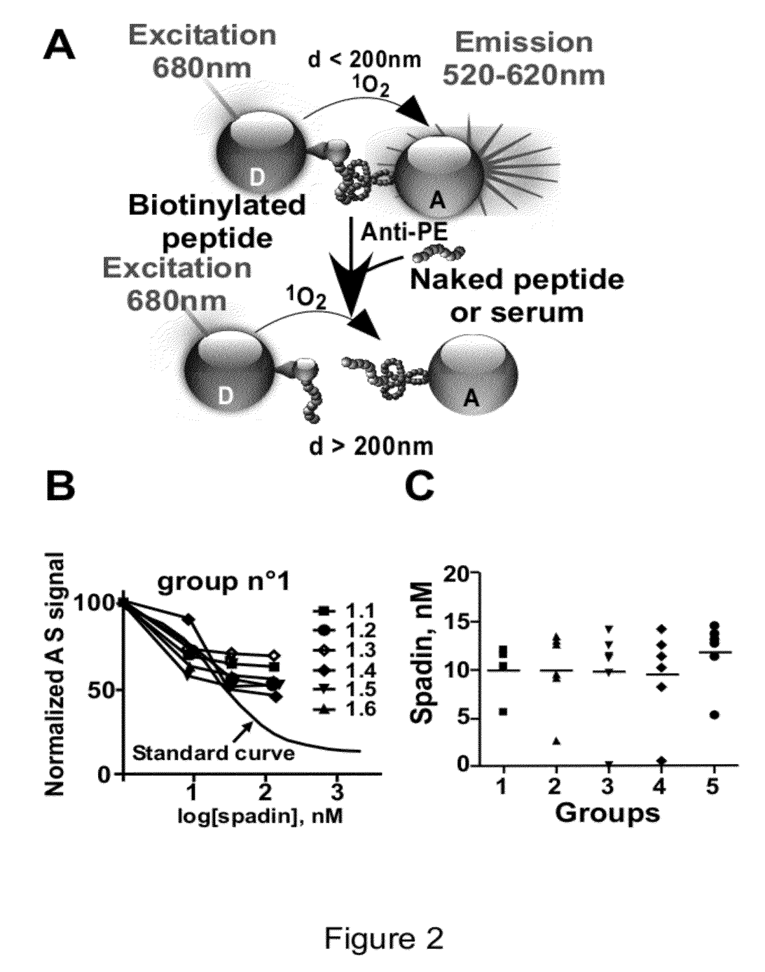

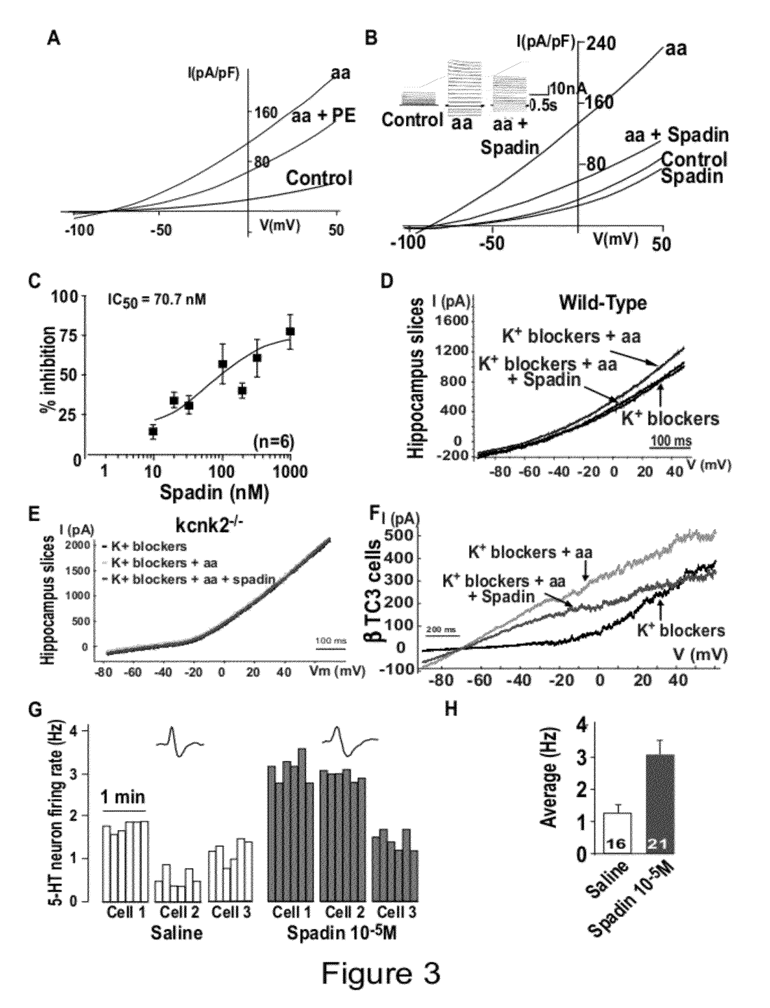

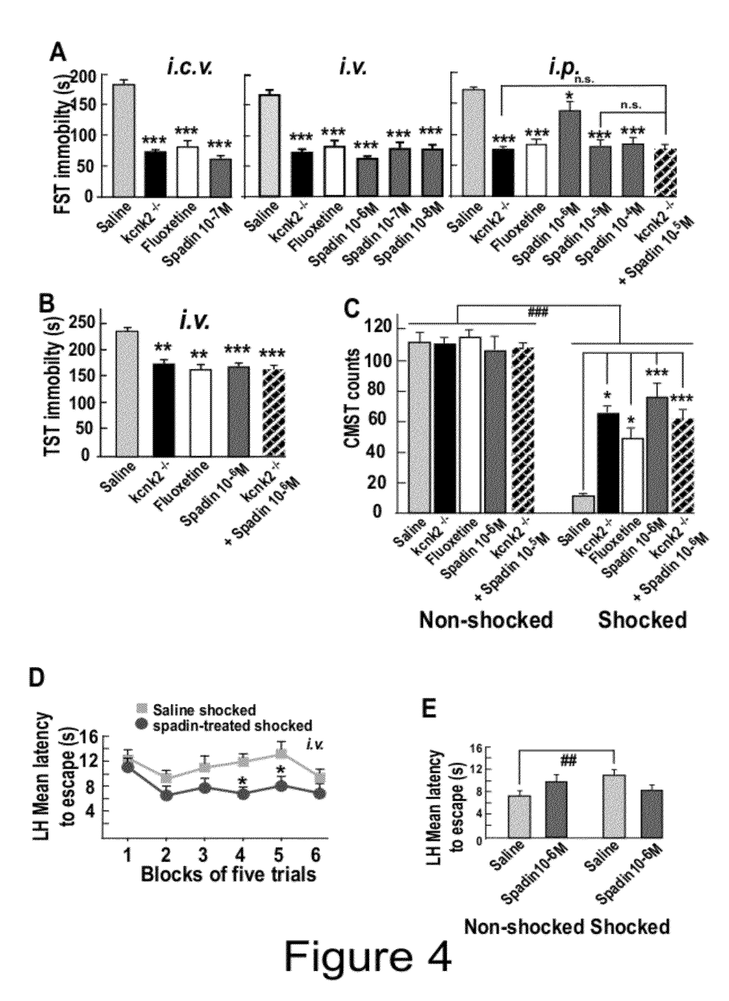

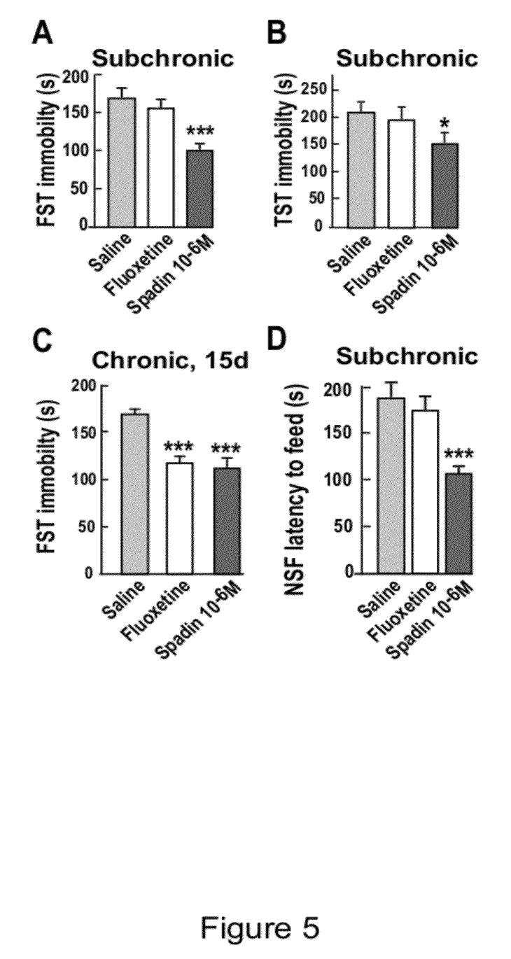

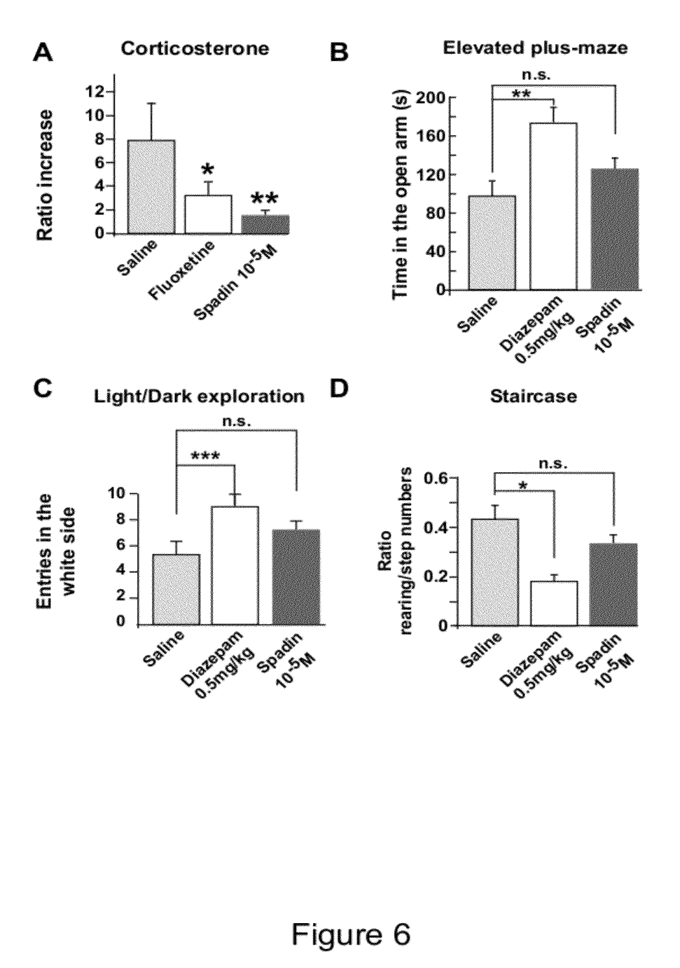

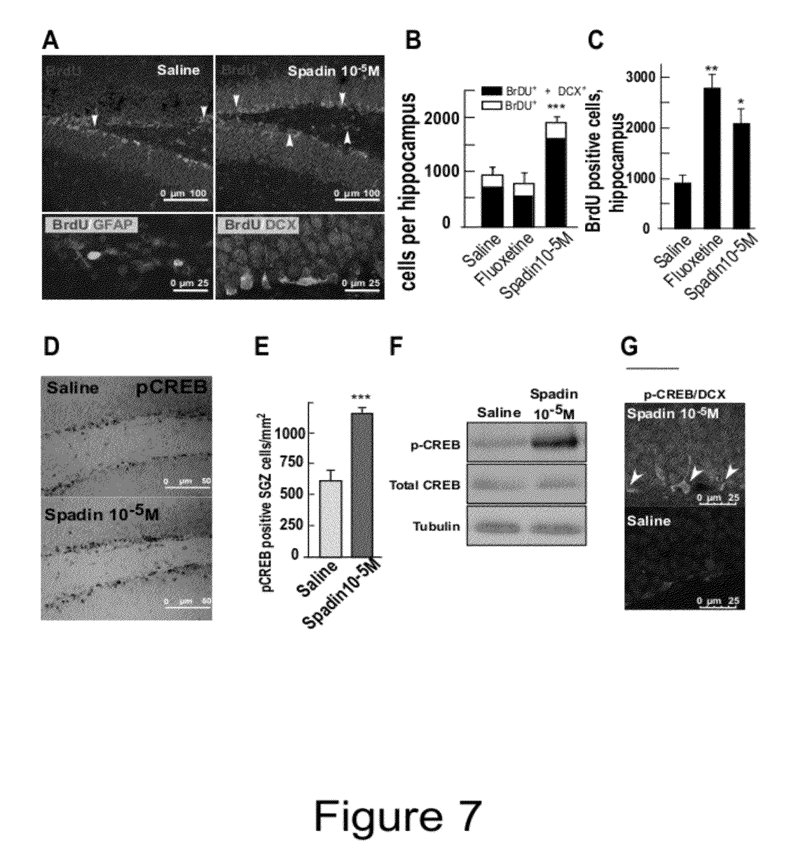

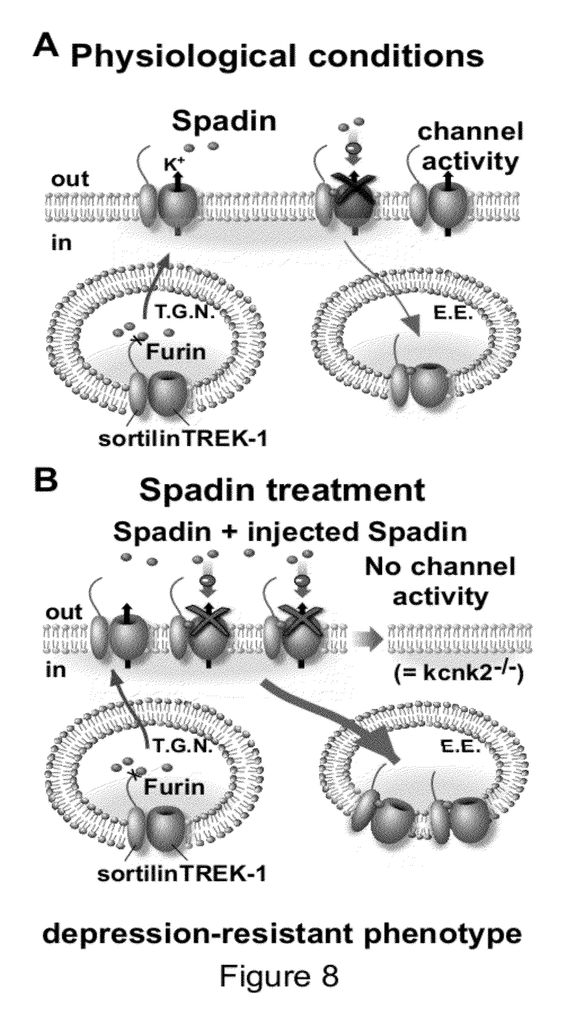

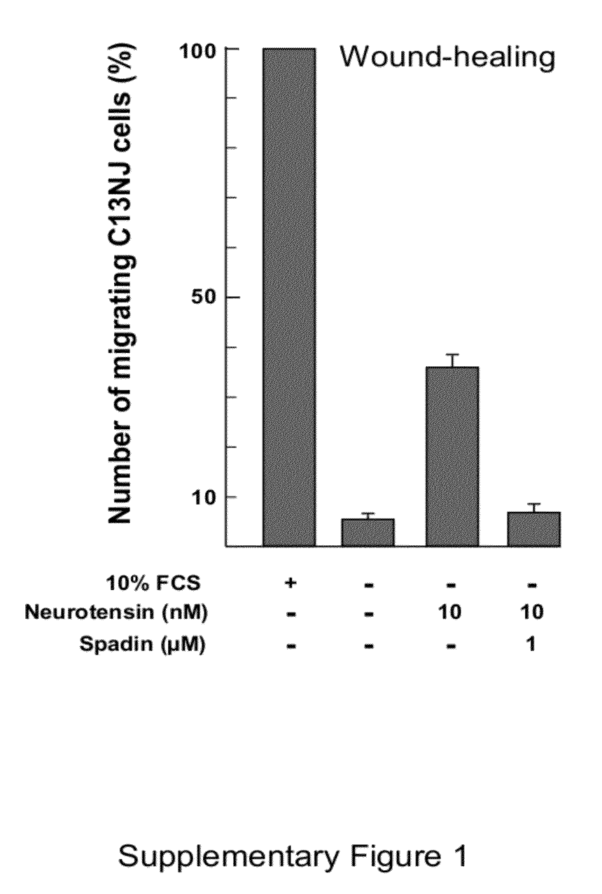

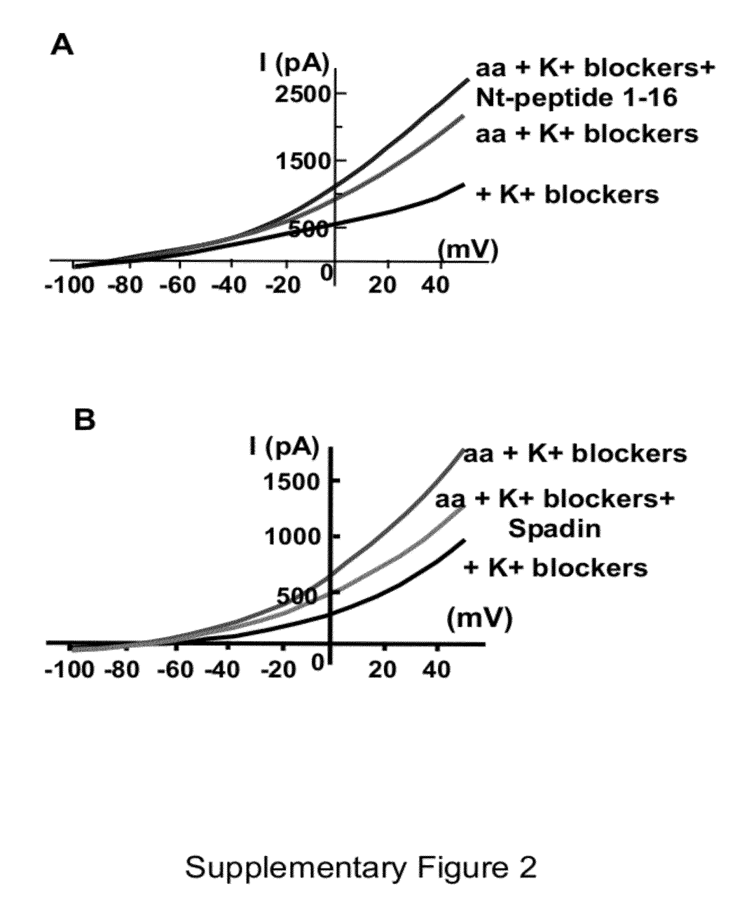

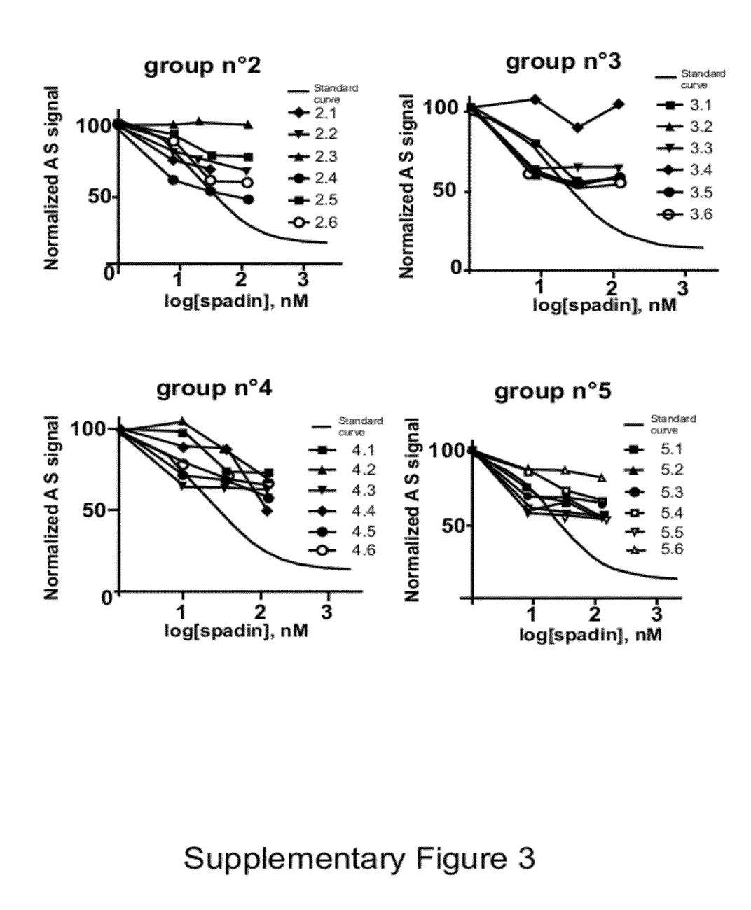

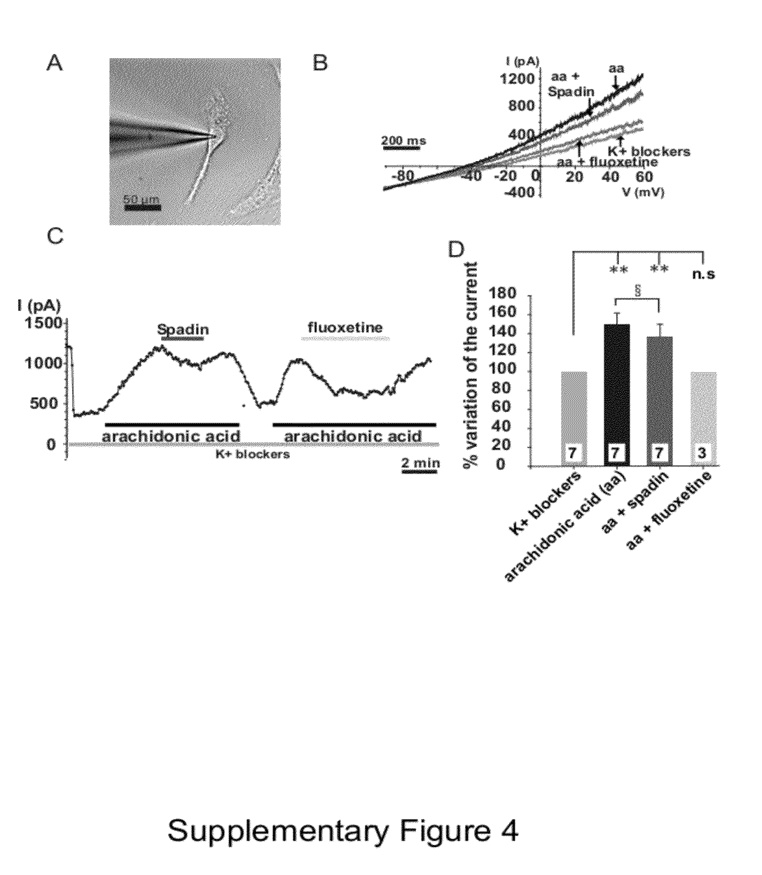

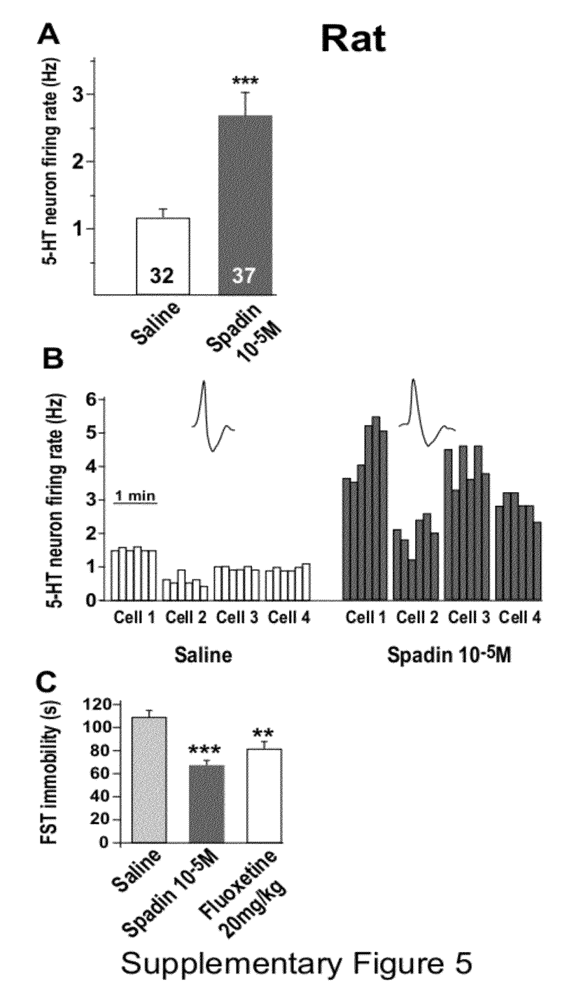

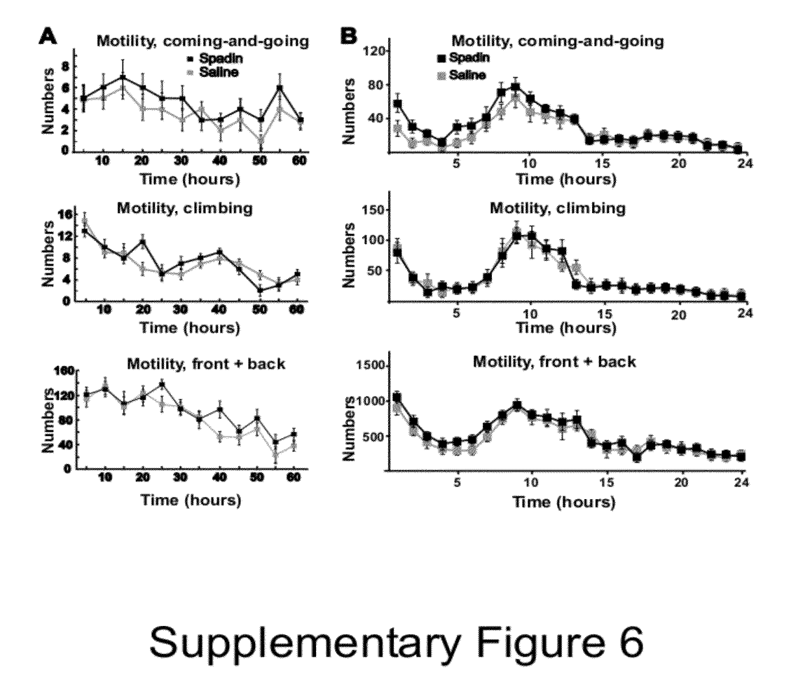

A, Principles of AlphaScreen™ technology. Donor and Acceptor microbeads can be coated with target-specific antibody, proteins or secondary reagents (streptavidin, glutathione, nickel). A signal is produced when the AlphaScreen Acceptor (A) and Donor (D) beads are brought into proximity by a molecular interaction occurring between the binding partners captured on the beads. Laser excitation at 680 nm causes ambient oxygen to be converted to the singlet state by photosynthetizers on the Donor bead. These react with chemiluminescent agents on the Acceptor bead only when the latter is in close proximity, emitting light at 520-620 nm. Here, we illustrate a competition protocol between seric propeptide (PE) and interacting donor beads (D) coupled-biotinylated spadin (b-spadin) with antibodies anti-propeptide (anti-PE) coupled on acceptor beads (A). B, An example of competition curve obtained with one group (n° 1) of 6 mice (1.1 to 1.6) among 5 different groups (other curves are presented in the Supplementary FIG. 1). Values obtained are compared to the standard curve. C, Seric concentrations of the full length propeptide calculated for the 5 groups from competition experiments as shown in B.

FIG. 3. Effects of Spadin on the TREK-1 channel activity.

A-B, Whole-cell currents measured in COS-7 transfected cells in presence of potassium blockers (K⁺ blockers, 10 mM tetraethyl ammonium (TEA), 3 mM 4-aminopyridine (4-AP), 50 nM charybdotoxin, 10 μM glibenclamide, 100 nM apamin). Cells were clamped at −80 mV and voltage changes were either applied by ramp from −100 to 50 mV, 1 s in duration (A, B main panel) or by 10 mV steps from −100 to 40 mV, 1.5 s in duration (B inset). Currents were recorded after TREK-1 activation by 10 μM arachidonic acid (aa) and aa+propeptide (PE, 500 nM) (A) or aa+Spadin (100 nM) (B). Native currents were recorded in absence (Control) and in presence of spadin (Spadin 100 nM, B). Peptides were applied via the bath medium. C, Dose-dependent spadin inhibition of TREK-1 currents, IC₅₀ value at 0 mV is of 70.7 nM. Currents were measured in presence of 10 μM aa. D-E, Native currents recorded in the presence of K⁺ blockers after stimulation by 10 μM of aa on CA3 pyramidal neurons from hippocampus slices in wild-type mice (D) or in kcnk2 deficient mice (kcnk2^{-/-}) (E) in the presence or the absence of spadin (1 μM). Currents were elicited by a ramp from −100 mV to 50 mV. F, Native currents on β-TC3 cell line in similar experimental conditions as (D-E). G-H, Effect of spadin on the firing rate of DRN 5-HT neurons. Spadin (10⁻⁵ M in a 100 μl bolus) or its vehicle was i.p. administered. Recordings started 30 min after the injection, and were performed for a maximal duration of 210 min thereafter. G, Main panel: Samples of “descents” performed along the DRN, showing typical integrated firing rate histograms in a vehicle- (left panel) or in a spadin-treated (right panel) animal. Each cluster represents the electrical activity of one neuron, each bar representing the average number of recorded action potentials per 10 s. Insets, examples of action potential waveforms of 5-HT neurons. H, 5-HT neuron firing activity, calculated on the basis of all the cells recorded within the successive tracks performed along the DRN. Values at the bottom of each column indicate the total number of neurons recorded (n=4 mice in both groups).

FIG. 4. Acute antidepressant effects of Spadin

A-E, Acute treatments: Spadin (10⁻⁴ to 10⁻⁸ M) or Fluoxetine (3 mg/kg) or Saline solutions were injected 30 min before the test in wild-type and Kcnk2^{-/-} mice (A-B-C). A, Forced Smimming Test (FST, n=10 per group), spadin-treated mice had a shorter time of immobility comparable to those obtained with kcnk2^{-/-} or fluoxetine-treated mice, whatever the way of spadin administration: intracerebroventricular (i.c.v., n=14 per group) (one way ANOVA, F_{3,55}=79.53, ***P<0.001 versus saline-treated mice), intravenous (i.v., n=8 per group except for fluoxetine and Kcnk2^{-/-} groups, n=6) (one way ANOVA, F_{5,43}=26.27, ***P<0.001 versus saline-treated mice) or intraperitoneal (i.p., n=10 per group except for kcnk2^{-/-}, n=5) (one way ANOVA, F_{3,34}=40.58, *P<0.05, ***P<0.001 versus saline-treated mice). B, Tail Suspension Test (TST, n=15 for saline and spadin groups, and n=9 for fluoxetine and kcnk2^{-/-} groups) i.v. spadin-treated mice had a shorter immobility score comparable to those obtained with kcnk2^{-/-} or fluoxetine-treated mice (one way ANOVA, F_{3,47}=11.40, **P<0.01, ***P<0.001 versus saline-treated mice). C, Conditioned Motility Suppression Test (CMST, n=10 per group). Two way ANOVA showed significant effects of shocks (F_{1,62}=254.1, P<0.001), treatment (F_{3,62}=3.87, P<0.01) and an interaction between these two factors (F_{3,62}=8.83, P<0.001). ^{###}P<0.01 versus non-shocked mice. In the shocked groups, spadin treatment reversed the freezing state induced by the shock training in saline-treated mice (78±7 vs 14±2 counts, respectively). This effect was stronger than those observed for kcnk2^{-/-} or fluoxetine-treated mice (one way ANOVA, F_{3,39}=10.87, *P<0.05, ***P<0.001 versus saline-treated mice). Counts are the number of squares crossed plus the number of climbings. D-E, Learned Helplessness test (LH), n=12 per group). Shocked spadin-treated mice showed shorter escape latencies than saline-treated mice. Two-way ANOVA showed significant effect for treatment (F_{1,110}=7.93, P=0.01) and for assay (F_{5,110}=3.56, P=0.005, *P<0.05 in shocked groups). D, Mean escape latencies ±SEM averaged in 6 blocks of 5 trials, and E, Mean overall latency ±SEM to escape across trials 1-30 as a function of spadin treatment. Two-way ANOVA (shocks X treatment) showed an interaction between these two factors (F_{1,44}=6.9, P=0.012). ^{##}P=0.007 for non-shocked saline-treated mice versus shocked saline-treated mice.

FIG. 5. Subchronic and chronic antidepressant effects of Spadin

Subchronic treatments: Spadin (10⁻⁶ M), Fluoxetine (3 mg/kg) or Saline solutions were i.v. injected in a 100 μL bolus once a day for 4 successive days before the test. In chronic treatments, spadin (10⁻⁶ M) and fluoxetine (1 mg/kg) were i.v. injected in a 100 μL bolus once a day for 15 successive days. For each test there were 8 animals per group. A, In FST (one way ANOVA, F_{2,23}=26.08, ***P<0.001) and B, TST (one way ANOVA, F_{2,24}=9.8, *P<0.05 versus saline-treated mice), spadin induced similar behaviours than those obtained with the acute treatment, whereas fluoxetine was without effect [30]. C, In FST, chronic treatment with spadin or fluoxetine significantly reduced the time of immobility (one way ANOVA, F_{2,26}=25.08, ***P<0.001 versus saline-treated mice). D, NSF paradigm: at the end of the 4-day treatment, animals were food deprived for one day and then measured for their latency to feed. Spadin treatment significantly reduced the latency to feed when compared to saline or fluoxetine treatments. (t-test, ***P<0.001 versus saline-treated mice). In all graphs, data are expressed as means±SEM.

FIG. 6. Effect of Spadin on stress and anxiety behaviors

A, Decreased stress-induced serum levels of corticosterone in mice treated with spadin. We compared serum corticosterone concentrations (ng/ml) sampled in the morning in mice acutely treated with spadin (i.v, 10⁻⁶M), saline or fluoxetine (i.p., 3 mg.kg) 30 min after a 10-min tube restraint (n=10 per group). Data are expressed as increase of the ratio corticosterone levels 30 min after stress over basal corticosterone levels 30 min before restraint (one way ANOVA, F_{2,27}=18.30, *P<0.05, **P<0.01 versus saline-treated mice). B, Effect of spadin (i.p, 10⁻⁵ M) and diazepam (i.p., 0.5 mg/kg) on time spent in the open arms (s) of the elevated plus-maze (n=10 per group, one way ANOVA, F_{2,27}=8.75, **P<0.001 versus saline-treated mice). C, Effect of spadin (i.p, 10⁻⁵ M) and diazepam (i.p., 0.5 mg/kg) on the total number of entries in the aversive white side in the light/dark transition test (n=10 per group, one way ANOVA, F_{2,53}=7.65, ***P=0.001 versus saline-treated mice). D, Influence of spadin (i.p, 10⁻⁵ M) and diazepam (i.p., 0.5 mg/kg) on mouse performance in the staircase test. Data are presented as the ratio of number of rearings over the number of ascended steps (n=10 per group, one way ANOVA, F_{2,44}=4.86, *P<0.05 versus saline-treated mice). In the three tests, mice were injected with either spadin or diazepam 30 min before the test. In all graphs, bars indicate SEM.

FIG. 7. Effects of Spadin on neurogenesis and CREB activation

A-B, Spadin increased neurogenesis A, Top, Representative photomicrographs of BrdU-labeled neurons in the dentate gyrus of the mouse hippocampus treated either with saline or with spadin (i.v., 10⁻⁶ M) for 4 days. Bottom, double labeling of BrdU-labeled neurons either with GFAP (glial marker) or with DCX (neuronal precursor marker), showing a co-localization only with DCX, and not with GFAP, B, Quantitation of BrdU positive cells of hippocampus treated with saline, fluoxetine or spadin (10⁻⁵ M) for 4 days. 85% of BrdU-labeled cells were positive to DCX. Data are number of BrdU⁺ or DCX⁺ cells in mouse hippocampus (n=5), (F_{2,53}=35.27; ***P<0.001 versus saline). C, Quantitation of BrdU positive cells of hippocampus treated with saline, fluoxetine or spadin (10⁻⁵ M) for 15 days. (n=5), (F_{2,53}=19.43; *P<0.05, **P<0.01 versus saline) D-G, Enhanced spadin treatment-induced CREB activation in the hippocampus, as assessed by measuring phosphoCREB (pCREB) immunoreactivity. D, Immunological distribution of pCREB in the mouse hippocampus after a 4-day i.v treatment. pCREB is phosphorylated in the cells near the subgranular zone (SGZ). E, Quantification of pCREB positive cells/mm² in hippocampal SGZ (n=5), (t test; ***P<0.001). (F), Western blot analysis of pCREB level in hippocampus treated with saline or spadin (10⁻⁵ M). (G), Double immunofluorescent staining (examples are indicated by arrows) for pCREB and DCX positive hippocampal neurons treated with saline or spadin (10⁻⁵M).

FIG. 8. Schematic model of TREK-1 regulation by NTSR3/Sortilin and Spadin

In physiological conditions A, the concentration of spadin that would be released from vesicles of the Trans Golgi Network (TGN) is not sufficient to completely abolish the channel activity, by internalization via Early Endosome (E.E.) vesicles, direct blockade, or both. Conversely, under spadin treatment B, the amount of spadin is sufficient to internalize all channel molecules and consequently to abolish the channel activity.

FIG. 9/Supplementary FIG. 1: C13NJ wound-healing assay

A cell-free zone was created within a semi-confluent monolayer with a pipette tip. We analyzed by time-lapse microscopy how cells repopulated the cell-free zone. The 100% of migrating cells was calculated by stimulation of cell migration with 10% of Fetal Calf Serum (FCS).

FIG. 10/Supplementary FIG. 2: Comparative effects of N-terminal propeptide fragment Gln1-Arg16 and spadin on TREK-1 channel activity: in COS-7 transfected cells.

A, After 90 sec of application, the N-terminal propeptide fragment Gln1-Arg16 (Nt-Peptide 1-16) was unable to inhibit the current increase induced by a 10 μM arachidonic acid (aa) application (the current value continued to increase) B, whereas in the same experimental conditions spadin inhibited the aa-induced increase.

FIG. 11/Supplementary FIG. 3: Alpha Screen™ assays.

Competition curve obtained with four groups (n° 2 to 5) of 6 mice. Values obtained are compared to the standard curve.

FIG. 12/Supplementary FIG. 4: Effects of spadin on cultured pyramidal neurons from hippocampus

A, Cultured pyramidal neuron from hippocampus, with on top, the patch-clamp pipette. Neurons were chosen according to their morphology. B, Current recorded in response to ramps of potential obtained in the various conditions indicated (aa: arachidonic acid: 10 μM, Spadin: 1□μM, Fluoxetine: 20 μM). Neurons were recorded in the whole-cell configuration of the patch-clamp technique. In voltage-clamp, ramps of potential from −90 mV to 70 mV were applied every 10 sec. The % variation of the current amplitude was always measured at −50 mV. The currents were recorded in response to such ramps in control conditions and in the presence of a cocktail of potassium blockers suitable to isolate the TREK currents (10 mM tetraethyl ammonium (TEA), 3 mM 4-aminopyridine (4-AP), 50 nM charybdotoxin, 10 μM glibenclamide, 100 nM apamin). In the presence of the potassium blockers, the remaining current was increased by about 50% by 10 μM arachidonic acid in 7 out of 70 recorded neurons (B-C), suggesting the activation of a TREK current. This acid-arachidonic evoked current (putative TREK current) was 49.7±16.38% (n=6) blocked by 1 μM spadin and fully blocked by 20 μM fluoxetine (B-D). The blocks were reversible. C, Time course of spadin and fluoxetine effects. Applications are indicated by the horizontal bars.

FIG. 13/Supplementary FIG. 5: Antidepressant effect of spadin in rats

A-B, Effect of spadin on the average DRN 5-FIT neuron firing rate. Spadin (10⁻⁵ M in a 500 μl bolus) or its vehicle (saline) was i.p. administered. Recordings started 30 min after the injection, and were performed for a maximal duration of 210 min thereafter. A, 5-HT neuron firing activity, calculated on the basis of all the cells recorded within the successive tracks performed along the DRN. Values at the bottom of each column indicate the total number of neurons recorded (n=4 rats in both groups). In saline-injected rats, we found a value of 1.18±0.13 Hz whereas it reached 2.66±0.36 Hz in the group treated with spadin (10⁻⁵M in a 500 μl bolus, i.p.) [one-way ANOVA, F(1, 68)=13.06, p<0.001]. This effect corresponds to an increase of 125%, a value strikingly similar to that of 146% observed in the mice experiments. B, Again, and as illustrated, several neurons found in spadin-injected rats discharged at up to 4, 5 or even 6 Hz, whereas most of the frequencies found in the saline group were in a normal (0.8-1.6 Hz) range. Samples of “descents” performed along the DRN, showing typical integrated firing rate histograms in a vehicle-(left panel), or in a spadin-treated (right panel), animal. Each cluster represents the electrical activity of one neuron, each bar representing the average number of recorded action potentials per 10 s. Insets, examples of action potential waveforms of 5-HT neurons. C, Acute antidepressant effects of Spadin in Forced Swimming Test (FST). Spadin (10⁻⁵ M), Fluoxetine (20 mg/kg) or Saline solutions were injected 30 min before the test in rats (n=10 per group). Spadin-treated rats had a shorter time of immobility comparable to those obtained in fluoxetine-treated animals (one way ANOVA, F_{2,26}=16.66, ***P<0.001, **P<0.01 versus saline-treated rats).

FIG. 14/Supplementary FIG. 6: Mouse locomotion activity

To determine whether spadin induced a change in locomotor activity, mice (n=8 per group) were injected with the saline solution or spadin (10⁻⁵M in 100 μl bolus, i.p.) 30 min before starting the test session. Locomotor activity was monitored individually for 24 hours using an infrared photobeam activity monitoring system (Imetronic, Pessac, France), which measured consecutive horizontal beam breaks. Testing was in transparent plastic cages (43×20×20 cm³) with fresh bedding in a grid of 8 cm horizontal infrared beams. Locomotor activity was defined as breaking of consecutive photobeams. Mice were given one hour habituation session before being treated with spadin or saline. Movements were recorded and totalized for each 5 min period during the first hour and then by 10 min time section for the next 23 hours. 6 periods were pooled to obtain data for one hour of time. Different movements were monitored: the coming-and-going between the back and the front of the cage, climbing, and other movements in the back or the front of the cage. Data are the mean value of 8 animals per condition, bars represent SEM. Mice were kept under standard laboratory conditions: 12:12 light-dark cycle with free access to food and water during the experiment. There was no significant difference in locomotor activity between spadin- and saline-treated mice within 1 or 24 hours after the drug injection.

### DETAILED DESCRIPTION OF THE INVENTION

Here, we validate the antidepressant effects of spadin (also called “propeptide”), a secreted peptide derived from the propeptide generated by the maturation of the neurotensin receptor 3 (NTSR3/Sortilin) and acting through TREK-1 inhibition.

This study identifies spadin as the first peptidic antagonist of the TREK-1 channel, and illustrates its potent antidepressant properties by using biochemical, electrophysiological and behavioural approaches. Spadin is a secreted peptide derived from the propeptide released from the precursor form of NTSR3/Sortilin. Here, we show for the first time that the propeptide is present into blood circulation, and is able to inhibit currents mediated by the TREK-1 channel. Due to higher affinity and for a better efficacy, this study was mainly focused on the use of spadin. Using TREK-1 deficient mice and animal models of depression, our laboratory has recently identified the TREK-1 channel as a new target for depression and its blockers as potential antidepressant drugs [1]. With the identification of spadin as an antagonist of TREK-1, this work validates the TREK-1 channel as a good target for the development of drugs for the treatment of depression [1, 40]. In humans, the Star*D study has reported the functional role of this particular potassium channel in mood regulation and in resistance to antidepressant treatments [4], strengthening the idea that TREK-1 represents an attractive pharmacological target for the development of new types of antidepressant drugs.

This is of high relevance since depression is a devastating illness that affects ˜17% of the population at some point in life, resulting in major social and economic consequences [41]. Designing effective treatments for this serious disorder is challenging, in part because unravelling the exact changes that lead to this psychiatric disorder is particularly difficult. In addition to the inherent complexity of the disease itself, it is not clear how antidepressant drugs work. Most antidepressants increase levels of the monoamine serotonin (5-HT) and/or noradrenaline (NA), suggesting that biochemical imbalances within the 5-HT/NA systems may underlie the pathogenesis of this disorder [17, 27, 39]. To date, the mainstay of antidepressant treatments is constituted by selective serotonin reuptake inhibitors, which inhibit the 5-HT reuptake pump. Although antidepressant treatments significantly improve the therapeutic outlook for depressed patients, there are still too many patients who do not respond to initial treatments. In the case of response, side effects are often observed, as well as a delay in the onset of therapeutic efficiency and/or a partial rather than a full remission. Spadin, which is a natural peptide, may alleviate these problems and become a strong candidate to develop new efficient and fast-acting antidepressant treatments. The first result of this work is the identification of the NTSR3/Sortilin receptor as a novel TREK-1 partner protein. It interacts physically and functionally with TREK-1 to modify its cell surface expression. NTSR3/Sortilin is a member of the Vps10p-domain receptor family, which is expressed in several tissues, including the brain. The interaction between NTSR3/Sortilin and the N-terminal portion of the precursor form of the nerve growth factor (pro-NGF) and the brain-derived neurotrophic factor (pro-BDNF) represents a key event in the process that controls neurotrophins-mediated cell survival and death in developing neuronal tissue and post-traumatic neuronal apoptosis [42]. NTSR3/Sortilin is involved in the sorting of BDNF [43]. In regard to depression (for review see [44]), it is well known that exogenous delivery of neurotrophic factors, such as BDNF and or neurotrophin 3 (NT-3) promotes the function, sprouting and regrowth of 5-HT neurons in the rat brain. Infusions of BDNF into the DRN produced an antidepressant effect, as evaluated by several learned helplessness paradigms. Environmental stressors induce depression and decrease BDNF mRNA, whereas antidepressants increase BDNF mRNA in the brain via 5-HT_{2A} and β-adrenoreceptor subtypes. Since we observed an activation of 5-HT neurons by spadin, it would be important to measure the influence of spadin both on protein and mRNA levels of BDNF in order to determine whether its action is correlated with the modulation of neurotrophin pathways. NTSR3/Sortilin and spadin interact with the TREK-1 channel as shown by immunoprecipitation of TREK-1 and NTSR3/Sortilin from COS-7 cells co-expressing both proteins. TREK-1 and NTSR3/Sortilin are also colocalized in mouse cortical neurons.

This work identifies a new function for spadin as a peptidic antagonist of the TREK-1 channel. Until now, the full spadin “propeptide” (1-44) ¹QDRLDAPPPPAAPLPRWSGPIGVSWGLRAAAAGGAFPRGGRWRR⁴⁴ (SEQ ID NO: 2), which contains the active spadin (¹²APLPRWSGPIGVSWGLR²⁸, SEQ ID NO: 1) was known to display two principal functions: (1) it binds the mature form of NTSR3/Sortilin, hindering ligands to access the binding site of the receptor, and (2) it antagonizes the effects of neurotensin on microglial cell migration [9, 14]. To our knowledge, the propeptide has no additional protein target. Here, we determined that spadin, but not its N-terminal fragment Gln1-Arg16, displays identical binding and activity properties as those of the full propeptide on the neurotensin system. Spadin binds specifically to TREK-1 with an affinity of 10 nM. Electrophysiological studies show that spadin efficiently blocks the TREK-1 activity in COS-7 cells, cultured pyramidal neurons as well as in CA3 hippocampal neurons in brain slices of wild-type mice and not in kcnk2^{-/-} mice, suggesting a specific effect of spadin on the TREK-1 channel.

Finally, our data point out spadin as the first peptidic and fast-acting antidepressant. Considering the blocking effect of spadin on TREK-1 channels, we have analyzed in vivo its potential antidepressant effects. In behavioral tests (FST, TST and CMST), predicting an antidepressant response [24], spadin-treated mice show a resistance to depression as do kcnk2^{-/-} mice [1]. This antidepressant phenotype is even more marked in the LH and NSF tests, which are considered as classical “rodent models of depression” [13, 31]. The antidepressant effect of spadin is not specific to mice since it has also been observed in rats using the FST test and in vivo 5-HT neuron firing recordings. More importantly, our results indicate that molecular, biochemical and behavioral changes, that have previously been specifically linked to long-term chronic treatment with SSRIs, are already present as soon as 4 days when using i.v. spadin administration. The fast-acting antidepressant potential of spadin, observed in vivo in FST and TST tests is further confirmed by its ability to activate CREB function and neurogenesis in the adult mouse hippocampus after a subchronic treatment. It is now well stated that antidepressants share the common property to positively modulate cellular growth and plasticity in mood-related brain areas. Indeed, CREB activity and neurogenesis are considered as specific markers of antidepressant action [45], but have never been observed before 2 weeks of treatment when using classical antidepressants such as SSRIs. By binding to cAMP response element (CRE) sites, CREB mediates transcriptional responses to elevated levels of cAMP. CRE-mediated gene transcription is upregulated after chronic antidepressant treatment [48]. CREB upregulation activates downstream targets such as the brain-derived growth factor (BDNF) after antidepressant treatment by binding to CRE elements located in the promoter region of the BDNF gene [49]. Here, we show that a 4-day chronic treatment with spadin is able to enhance the pCREB/CREB ratio and consequently increases cell division and proliferation in the SGZ. In addition, the therapeutic potential of spadin appears to be specific of depression, in that it is unable to affect anxiety-related behaviors. This is in good agreement with the fact that TREK-1 deficient mice do not show an anxiety-resistant phenotype [1]. In contrast, both spadin and the deletion of the TREK-1 channel induce an hypoactivity of the hypothalamic-pituitary-adrenal axis when animals are exposed to stress.

As described in kcnk2^{-/-} mice [1], spadin leads to an in vivo increase in efficacy of 5-HT neurotransmission as evidenced by an increased firing activity of DRN 5-HT neurons. Even if the involvement of other aminergic systems in the pathophysiology of depression is certainly non-negligible, it remains that the facilitation of central 5-HT transmission constitutes the common property of all the antidepressant strategies, which have proved their efficiency. From a mechanical point of view, 5-HT_{1A} autoreceptor stimulation reduces DRN 5-HT neuronal firing and, consequently, 5-HT neurotransmission [28]. Inhibition of adenylate cyclase and activation of G-protein-coupled inwardly rectifying K+ channels (GIRK) are involved in this negative feedback [40]. The decrease in cAMP concentration (as a result of reduced adenylate cyclase activity) in 5-HT neurons is also thought to induce TREK-1 opening because of a consequent reduction of phosphorylation of Ser333 by PKA [16]. According to this model, spadin would induce a depolarization by closing TREK-1 channels and, as described for TREK-1 deficient mice [40], would therefore reduce the negative feedback on 5-HT neurons, resulting in increased 5-HT neurotransmission and in turn in antidepressant-like effects. Direct inhibition of TREK-1 by spadin may also contribute to enhanced 5-HT neuron excitability. Because (i) sortilin is the partner protein of the TREK-1 channel and (ii) both proteins are colocalized in 5-HT-enriched areas known to be involved in the pathophysiology of depression such as the prefrontal and cingulate cortice, amygdala, hippocampus, nucleus accumbens, dorsal raphe and hypothalamus [13], one may infer that spadin acts predominantly through a modulation of the brain 5-HT circuitry. Nevertheless, we cannot exlude that it can also involve other neurotransmission systems. Whatever the effector pathways though, the fact that spadin has no effects on kcnk2^{-/-} mice indicates that its action is first and foremost mediated by a modulation of TREK-1 channels.

Our results show that spadin induces an 80% internalization of these channels. We propose a model of regulation of TREK-1 expression and regulation by NTSR3/Sortilin receptor and spadin (FIG. 8). In physiological conditions (FIG. 8A), TREK-1 and NTSR3/Sortilin would associate in the TGN vesicle, where spadin is hydrolyzed by furin. When TGN vesicle merge to the plasma membrane, spadin would be released (as suggested in [14] and showed in FIG. 2B-C and Supplementary FIG. 3), and would bind either NTSR3/Sortilin, TREK-1 channel, or both. This would lead to the internalization of the TREK-1/Sortilin complex in early endosome and subsequently, to its degradation. In the presence of an excess of spadin given by administration (FIG. 8B), the rate of internalized complexes would be increased, resulting in a total disappearance of TREK-1 channels at the surface membrane. This prediction is supported by the fact that 80% of the ¹²⁵I-spadin bound on the TREK-1 transfected COS-7 cells are insensitive to an acid-NaCl wash (FIG. 1F), indicating that the complex spadin-TREK-1 is already internalized. The consequent loss of TREK-1 channel activity would lead to an antidepressant phenotype, as observed in TREK-1 deficient mice. However, overall the inhibitory action of spadin on TREK-1 function is likely the consequence of both its ability to induce channel internalization, and to its direct effect on the channel current.

To conclude spadin can be considered as a natural endogenous antidepressant and constitutes the first peptide identified as an antidepressant with a rapid onset of action. Due to these peculiar properties, spadin brings a new concept to address the treatment of depression. To date, spadin is also the first blocker of TREK-1 channel identified, which is not only of relevance in the field of depression, but also constitutes a useful tool to further understand the role of TREK-1 channels in other neurological pathologies. Finally, this work shows the development of a reliable method for dosing the propeptide and spadin by using AlphaScreen™ technology. The last point is crucial to use in the future spadin as a marker of depression by dosing spadin in the serum of depressed patients, and to help for setting clinical preventive protocols. Detecting and preventing the depression certainly could decrease the economic burden of this disease, which is estimated to be 44 billion dollars per year in the United States.

With the aim to use spadin as a marker of the depressive state in human, we already developed a method to measure its concentration in the blood of mice [57]. Since we possess both a monoclonal antibody directed against the peptide and a source of receptor (NTSR3) able to bind the propeptide, we are able to quantify its seric concentration in patients suffering or having suffered from depression compared to those of control (healthy) or anxious subjects. This can be achieved using for example Immunofluorescence, radio-immuno assay or radio-receptor assay or Elisa protocol or FRET technology, or other methods known to those of skill in the art.

The level of the spadin in the blood of depressive patients can be correlated to the level to a pathologic state (preliminary results obtained on mice samples have demonstrated the feasibility of the technique). Because a biomarker for the depression does not exist, the blood level of the propeptide could be the first tool to diagnostic the pathology. Moreover, when the propeptide or spadin or one of their fragments or derivatives will be used as depression biomarker, seric dosing techniques will be useful for physicians for establishing, monitoring and controlling the effective levels in each patients. The antibody is directed against the following sequence APLPRWSGPIGVSWGLR (SEQ ID NO: 1), then, this antibody is able to recognize both the full length and the partial sequences resulting from the maturation and/or the degradation of the 44 aa propeptide.

Consequently, an objective of the present invention is to measure the amount of the propeptide in the body fluids of depressive patients in order to correlate its level to the depression behavior (to determine whether the propeptide concentration is less or more important in depressive patient plasma in comparison with plasma of healthy subjects or anxious patients), to follow the evolution of the seric propeptide concentration after remission of depression or anxiety disorders, and then to use it as a pathological marker.

To this aim, we designed “spadin” by conserving the sequence 17-28 (¹⁷WSGPIGVSWGLR²⁸, SEQ ID NO: 3) from the full propeptide of 44 amino acids in which we added the sequence 12-16 (¹²APLPR¹⁶, SEQ ID NO: 4) in order to maintain conformational stress. This partial propeptide named “spadin” (¹²APLPRWSGPIGVSWGLR²⁸, SEQ ID NO: 1) was tested for its potential effects on TREK-1 channel regulation and for its validation as antidepressant drug in five behavioral models of depression. This will allow us to use the propeptide as biological marker of the depression disease.

The present invention relates to the use of the full propeptide of sequence SEQ ID NO: 2 or a fragment or derivative thereof, which maintains its capacity to bind the neurotensin (NT) receptor 3 (NTSR3, also called gp95/sortilin), as a diagnostic tool or biomarker of depression. In particular, for the diagnosis of patients suffering of having suffered from a psychiatric disease and/or for the monitoring of depressive patients after remission. By “fragment which maintains its capacity to bind the neurotensin (NT) receptor 3 (NTSR3, also called gp95/sortilin)” is meant for example a fragment of sequence SEQ ID NO: 1 or 3. By “derivative which maintains its capacity to bind the neurotensin (NT) receptor 3 (NTSR3, also called gp95/sortilin)” is meant for example an analogue of complete or partial propeptide wherein: at least one amino acid can be replaced by another one of the same family (aromatic, hydrophobe, basic, etc. . . . ), at least one natural amino acid (L aminoacid) can be replaced by the same amino acid under the form D, and/or at least one peptide bond between two amino acids can be replaced by a pseudo-peptide bond, thus generating variants which maintain their capacity to bind NTSR3 but that may be resistant to proteolytic attack of peptidases or proteases.

Thus, in one embodiment, the invention is directed to a method of diagnosing a psychiatric disease comprising analyzing a biological sample from a subject in need of diagnosis of the psychiatric disease for the expression of spadin, detecting and measuring the amount of spadin in the sample, and diagnosing psychiatric disease in the subject if an increase in spadin expression in the biological sample is detected compared to a control. In another embodiment, the invention is directed to a method of diagnosing psychiatric disease, such a depression, in a subject, comprising analyzing a biological sample from a subject in need of diagnosis of psychiatric disease for the expression of spadin, detecting and measuring the amount of spadin in the sample, and diagnosing psychiatric disease in the subject if a decrease in spadin expression in the biological sample is detected compared to a control.

Preferrably the psychiatric disease is depression. By “spadin” is preferably meant SEQ ID NO: 1 but also more generally speaking the full propeptide of sequence SEQ ID NO: 2 comprising the same or a biologically active fragment thereof, for example a fragment of sequence SEQ ID NO: 1 or 3. Thus in the above methods, the spadin to be detected and measured may be the full propeptide or any fragment thereof.

Preferably, the biological sample is a body fluid used in the above embodiments, which may be selected from the group consisting of blood, blood plasma, serum, bone marrow, stool, synovial fluid, lymphatic fluid, cerebrospinal fluid, sputum, urine, mother's milk, sperm, exudates and mixtures thereof.

The control, according to the invention, may be the amount of spadin in a sample from a “healthy” subject, i.e., a subject without a psychiatric disease, such as depression, or the control may be the amount of spadin in a sample from a subject with a known psychiatric disease, such as depression. One of skill in the art would understand the statistical protocols needed to arrive at the proper control values.

In one embodiment, the analyzing of spadin expression may comprise using a probe, such as an antibody that specifically binds spadin. In a preferred embodiment, such antibody binds SEQ ID NO:1. One of skill in the art would understand how to prepare such antibodies and to modify such antibodies to enhance specificity. Antibodies include monoclonal and polyclonal antibodies, fragments of antibodies, affinity matured antibodies and humanized antibodies. In another embodiment, the invention is a nucleic acid probe that hybridizes with spadin-encoding nucleic acid (DNA, cDNA or mRNA). In another embodiment, the analyzing of the biological sample for spadin expression comprises use of nucleic acid amplification. Those of skill in the art would know how to prepare nucleic acid probes and engage in nucleic acid amplification techniques, such as PCR, using methods well known in the art. One of skill in the art would also understand how to label such probes so as to detect the expression of spadin, according to the invention.

The above diagnostic methods may be used in combination with other methods of diagnosing psychiatric diseases. The other diagnostic methods may be based upon behavioural assessments according to methods well known in the art.

In another embodiment, the invention is directed to a method of monitoring subjects being treated for psychiatric disease, such as depression, comprising a) detecting and measuring the amount of spadin in a biological sample from a subject being treated for psychiatric disease and b) comparing the amount in a) to the amount of spadin in a control, wherein a change in the amount of spadin is an indication that the treatment is effective. The methods of detection and measuring in monitoring treatment includes the use of probes and of nucleic acid amplification techniques, as mentioned above in connection with methods of diagnosis. The frequency of monitoring may be determined by the attending physician based upon what is standard practice for psychiatric therapy.

In another embodiment, the invention is directed to a method of monitoring psychiatric diseased subjects in remission, comprising a) detecting and measuring the amount of spadin in a biological sample from a subject thought to be in remission for psychiatric disease and b) comparing the amount in a) to the amount of spadin in a healthy control, wherein when the amount in a) is similar to the amount in the healthy control, there is an indication that the subject is in remission. The methods of detection and measuring in monitoring remission includes the use of probes and of nucleic acid amplification techniques, as mentioned above in connection with methods of diagnosis. The frequency of monitoring may be determined by the attending physician based upon what is standard practice for psychiatric therapy.

In another embodiment, the invention is directed to a method of determining the course of psychiatric disease, such as depression, in a subject comprising: a) measuring at a first time, the amount of spadin in a biological sample from the subject; b) measuring, at a second time, the amount of spadin in a biological sample from the subject; and c) comparing the first measurement with the second measurement; wherein the comparative measurements determine the course of the psychiatric disease in the subject. This method may be of prognostic value and may guide the attending physician in prescribing therapy. The methods of detection and measuring required for determining the course of the disease, includes the use of probes and of nucleic acid amplification techniques, as mentioned above in connection with methods of diagnosis.

A “subject” according to the invention is a mammal, preferably a human.

In another embodiment, the invention is directed to a method of identifying a candidate compound for treating depression, the method comprising determining whether the candidate compound inhibits currents mediated by the TREK-1 channel, wherein when the candidate compound inhibits currents mediated by the TREK-1 channel, it is a candidate compound for treating depression. The details of in vitro and in vivo methods for determining whether a compound inhibits TREK-1 Channel activity include those described in the Examples, below.

In a further aspect, the present invention provides kits for use in the methods of the invention, comprising spadin and/or detectably labeled probes of the invention and instructions for their use. In one embodiment, the probe is an antibody specific for spadin, preferably a monoclonal antibody that binds SEQ ID NO:1. In another embodiment, the probe is a polynucleotide or primer pair that are detectably labeled. In a further embodiment, the probes/primer pairs are provided in solution, most preferably in a hybridization or amplification buffer to be used in the methods of the invention. In further embodiments, the kit also comprises wash solutions, pre-hybridization solutions, amplification reagents, software for automation of the methods, etc. The kit may also container a control, according to the invention and more than one container.

### EXAMPLES

### Materials and Methods

All experiments were carried out on 20-25 g male C57Bl/6J (Janvier France Breeding) and on TREK-1 deficient mice (kcnk2^{-/-}) according to policies on the care and use of laboratory animals of European Community legislation 86/609/EEC. The local Ethics Committee (CREEA Côte d'Azur) approved the experiments (protocol number NCE2008-08/09-0). The behavioral protocols are described in the Supporting File.

### Drugs and Chemicals

The propeptide named spadin, with the following amino acid sequence: APLPRWSGPIGVSWGLR (GenBank NM_{—}019972 for mouse, SEQ ID NO: 1), and the N-terminal fragment QDRLDAPPPPAAPLPR (SEQ ID NO: 5) were synthetized by Gencust (France). The stock solution was dissolved in distilled water at a concentration of 10⁻³ M and spadin solution was then diluted in NaCl 0.9% to reach the different treatment concentrations. The intracerebroventricular (i.v.) injection was performed under isoflurane anaesthesia. Mice were anesthetized by inhalation of 2% isoflurane mixed with 30% oxygen and 70% nitrous oxide. Spadin (5 was stereotaxically administered 30 min prior to the behavioral test by an injection needle that was lowered bilaterally into the lateral ventricle of the mouse positioned on a stereotaxic frame, by using the coordinates referred from Paxinos and Flanklin (related to bregma: AP: −0.46 mm, ML: 1.25 mm and DV: −2.25 mm). The injection needle was connected to a Hamilton syringe (10 μl) positioned in a micropump and delivering the drug solution at a rate of 1□μl/min for 5 min. Fluoxetine (TEVA Santé, France) and diazepam, diluted in NaCl 0.9% were used at the concentration of 3 mg and 0.5 mg per kg body weight, respectively, in intraperitoneal (i.p.) administration. BrdU (Sigma-Aldrich, St Quentin Fallavier, France) was diluted in Tris-buffered saline (0.1 M in NaCl 0.9%, pH:7.6). All other chemicals were from Sigma (St Quentin Fallavier, France). Stock solutions were prepared in H₂O except otherwise mentioned, frozen and diluted before the experiment. Arachidonic acid which was prepared at a concentration of 0.1 M under argon in 100% ethanol, glibenclamide 100 mM in dimethyl sulfoxide (DMSO) and fluoxetine 1.3 mM in glycerol.

### Cell Culture

COS-7 and C13NJ cells were cultured in DMEM supplemented with 10% FBS and 50 μg/ml gentamycin at 37° C. under 5% CO₂. β-TC3 cells were grown in RPMI 1640 supplemented with 2.5% FBS, 50 μM β-mercaptoethanol, 10 mM HEPES, 1 mM Sodium pyruvate and 50 μg/ml gentamycin. Cells were maintained at 37° C. under 5% CO₂.

Cortical neurons were prepared from 14 old mouse embryos whereas hippocampal neurons were prepared from new born mice. Briefly, dissected brain areas were dissociated and neurons were plated on polylysine-treated 35 or 60 mm dishes and maintained in culture in Neurobasal medium supplemented with B27, Glutamax and antibiotics for 2 to 3 weeks before to be used for electrophsysiological or biochemical experiments.

### Characterization of the Interaction Between NTSR3/Sortilin, Spadin and TREK-1

Experiments were performed using COS-7 cells (10⁶ cells per diameter 100 dish) transfected with TREK-1 (2 μg/dish) in the presence or in the absence of NTSR3/sortilin (2 μg/dish) using the DEAE-dextran protocol.

Immunoprecipitation

Cortical neurons or COS-7 cells transfected with TREK-1 and NTSR3/Sortilin were lysed in 20 mM Tris-HCl pH 7.5, 50 mM NaCl, 50 mM NaF, 30 mM sodium pyrophosphate, 5 mM EGTA, 10% glycerol, 1% Triton X100, 1 mM PMSF, 1 mM Na3VO4, 5 μg/ml aprotinin (lysis buffer) for 1 h at 4° C. Solubilized proteins were clarified by centrifugation at 15,000×g for 15 min at 4° C. Supernatants were immunoprecipitated by using either the rabbit polyclonal anti-NTSR3 antibody (1:250) (Alomone) or the rabbit polyclonal anti-TREK-1 antibody (1:250) (Alomone) in the presence of 40 μl protein-A Affarose (Interchim) overnight at 4° C. Protein complexes were recovered by centrifugation at 15,000×g for 5 min at 4° C. and washed twice with the lysis buffer. Immunoprecipitates were resuspended in SDS buffer, separated by SDS-PAGE, transferred onto nitrocellulose and revealed either with anti-NTSR3 or with anti-TREK-1 (1:1000). Bound antibodies were visualized using HRP-conjugated goat anti-rabbit IgG TrueBlot.

Sub-Cellular Fractionation

Plasma membranes were prepared from COS-7 cells transfected with TREK-1 alone or with NTSR3/Sortilin according to the protocol described by Clancy and Czech [50]. 30 μg of crude homogenates or purified plasma membranes were submitted to Western blot analysis using the rabbit polyclonal anti-TREK-1 antibody (1:500). Alternatively, plasma membranes of COS-7 cells transfected with TREK-1 alone or with NTSR3/sortilin were labeled with 0.5 mg/ml Sulfo-NHS-SS-Biotin for 30 min at 4° C. Cells were recovered with the lysis buffer used for immunoprecipitation for 1 h at 4° C. and solubilized proteins were clarified by centrifugation at 15,000 g for 15 min at 4° C. before to be precipitated using streptavidin-agarose overnight at 4° C. Protein complexes were recovered, separated by SDS-PAGE and submitted to Western blot analysis as described above.

Spadin Iodination

Spadin (2 nmol) was iodinated with ¹²⁵INa (0.5 nmol) using lactoperoxidase as oxidant. Monoiodinated spadin (on Tyr0) was purified by HPLC using a Waters apparatus equipped with a RP18 Lichrosorb column. Elution was carried out at a flow rate of 1 ml/min with a linear gradient of increasing concentration of acetonitrile in water containing 0.1% TFA from 30 to 60% in 36 min. The iodinated peptide was eluted at 24 min.

Binding Assays

For competition experiments, homogenates from TREK-1 transfected COS-7 or C13NJ cells were incubated with 0.2 nM ¹²⁵I-spadin or ¹²⁵I-NT (200,000 cpm in 250 μl) iodinated and purified as previously described [51]. Incubations were performed in 50 mM Tris-HCl, pH 7.4 containing 0.1% BSA in the presence of increasing concentrations of non-radioactive spadin or NT (10⁻¹⁰ to 10⁻⁵M). Incubations were terminated by addition of 2 ml of ice-cold binding buffer followed by filtration through cellulose acetate filters (Sartorius, Göttingen, Germany) and washing twice with 2 ml of ice-cold buffer. Radioactivity on filters was counted with a gamma-counter.

Wound-Healing Assay

A cell-free zone was created within a semi-confluent monolayer of microglial culture by scratching cells off with a pipette tip. We analyzed by time-lapse microscopy how cells repopulated the cell-free zone, as already described [17].

Internalization

Cells, grown on 12 mm multiwell-dishes, were incubated with 0.2 nM ¹²⁵I-spadin for various times at 37° C. in an Earle's Tris-Hepes buffer (cell binding buffer). Incubations were terminated by washing cells twice with the binding buffer or with the same buffer containing 0.5 M NaCl, pH 4, for 2 min to remove non sequestrated radioactivity (acid-NaCl wash). Cells were harvested with 1 ml of 0.1 N NaOH and counted in a gamma-counter. Non specific binding was determined in the presence of 10 μM unlabeled spadin.

TREK-1/NTSR3/Sortilin Colocalization Experiments

Hippocampal neurons were first washed for 5 min in Phosphate-Buffered Saline (PBS), then fixed with 4% paraformaldehyde in PBS for 20 min at room temperature. Coverslips were rinsed twice with PBS and incubated with 50 mM NH4Cl in PBS for 10 min to quench excess of free aldehyde groups. After 20 min in PBS containing 10% Horse Serum (HS), cells were labeled with a goat polyclonal anti-NTSR3/Sortilin (1/100) (Santa Cruz) and a rabbit anti-TREK-1 [16] (1/3000), for 1 h at room temperature in PBS containing 5% HS. Cells were rinsed three times in PBS, then incubated at room temperature in PBS containing FITC conjugated donkey anti-goat antibody (1/1000) and a Texas Red conjugated donkey anti-rabbit antibody (1/1000) in PBS containing 5% HS for 45 min. After two washes with PBS and one with water, coverslips were mounted on glass slides with mowiol for confocal microscopy examination.

### Electrophysiology

COS-7 Cells

All electrophysiological experiments were done on COS-7 cells seeded at a density of 20,000 cells/35-mm dish 24 h before transfection. Cells were transfected by the classical DEAE-dextran method with TREK-1-GFP plasmids (0.1 μg/μL). Cells were visualized 48-72 h after transfection using fluorescence.

The whole-cell patch-clamp technique was used to evaluate TREK-1 potassium channel current by using a RK 400 patch-clamp amplifier (Axon Instruments, U.S.A.), as previously described [52]. Currents were lowpass filtered at 3 kHz, digitized at 10 kHz using a 12-bit analog-to-digital converter. Patch-clamp pipettes were pulled from borosilicate glass capillaries and had a resistance of 1.8-3 MΩ. The bath solution contained (in mM): 150 NaCl, 5 KCl, 3 MgCl₂, 1 CaCl₂, and 10 HEPES, adjusted to pH 7.4 with NaOH; the patch pipette solution contained (in mM): 155 KCl, 3 MgCl₂, 5 EGTA, and 10 HEPES, adjusted to pH 7.2 with KOH. Cells were clamped at −80 mV and voltage changes were either applied by ramp (from −100 to 50 mV, 1 s in duration) or by step (from −100 to 40 mV, 1.5 s in duration). Cells were continuously superfused with a microperfusion system. The pipette capacitance was not subtracted from total membrane capacitance and there was no leak substraction. All experiments were done at room temperature (21° C. to 22° C.) and in the presence of a cocktail of potassium channel inhibitors (K⁺ blockers: 10 mM tetraethyl ammonium (TEA), 3 mM 4-aminopyridine (4-AP), 50 nM charybdotoxin, 10 μM glibenclamide, 100 nM apamin).

Pclamp software was used to analyze currents recorded in the whole-cell mode measured at 0 mV. Results are expressed as means±SD. To obtain the IC₅₀ value for dose-dependent inhibition, experimental data were fitted with a standard sigmoidal function.

β-TC3 Cells

Native currents elicited by these cells were recorded in the whole-cell configuration of the patch-clamp technique (as described for COS-7 cells) and in the presence of K⁺ blockers.

Brain Slices of Hippocampus

12-27 day old mice were anaesthetized with 1% halothane. Following decapitation, brains were rapidly removed and placed in cold phosphate/bicarbonate buffered solution (PBBS, 4° C.) composed of (mM): 125 NaCl, 2.5 KCl, 0.4 CaCl₂, 1 MgCl₂, 25 glucose, 1.25 NaH₂P0₄, 26 NaHC0₃, pH 7.4 when bubbled with 95% O₂/5% CO₂. Transversal 250 μm thick hypothalamic slices cut with a vibrating microtome (Microm, Francheville, France) were then transferred to an incubation chamber maintained at 34° C. in oxygenated PBBS. After one hour, slices were transferred to another incubation chamber at room temperature (22-25° C.) filled with PBBS containing 2 mM CaCl₂.

For current measurements using the whole-cell patch-clamp technique, brain slices were placed under a Nomarski microscope (Zeiss, Le Pecq, France) equipped with infrared video camera (Axiocam, Zeiss, Le Pecq, France) in a recording chamber superfused at a flow rate of 1 ml.min⁻¹ with HEPES solution containing (in mM): 140 NaCl, 5 KCl, 2 CaCl₂, 2 MgCl₂, 10 Glucose, 10 Hepes, pH 7.4. Pictures were taken by using a digital camera (Axiocam, Zeiss) connected to image-acquisition software (Axiovision). Recordings were made at room temperature (25±2° C.) using an Axopatch 200B (Axon Instruments, Foster City, Calif., USA). Patch clamp pipettes made from borosilicate glass capillary (Hilgenberg, Masfeld, Germany) had a resistance of 4-10 MΩ when filled with the internal solution containing (mM): 135 KCl, 5 NaCl, 2 MgCl₂, 5 EGTA, 10 Hepes (pH adjusted to 7.25 with KOH). Values of access resistance ranged from 12 to 20 MΩ and were not compensated. Measurements were made 2-3 min after obtaining the whole-cell to ensure dialysis. Changes of extracellular solution were obtained by a fast multi-barrel delivery system positioned close to the cell tested. Stock solutions were prepared in H₂O except otherwise mentioned, frozen and diluted before the experiment. Arachidonic acid which was prepared at a concentration of 0.1 M under Argon in 100% ethanol, glibenclamide 100 mM in dimethyl sulfoxide (DMSO) and fluoxetine 1.3 mM in glycerol.

Statistical significance between groups (average data expressed as mean±SEM, n=number of neurons) was tested using the Student's t-test or the ANOVA followed by t test, and were considered significant at P<0.05. Statistical analysis was done using SigmaPlot (Jandel) and Origin (Microcal) softwares.

Extracellular Unitary Recordings of DRN 5-HT Neurons

As previously described [1], single-barreled glass micropipettes (recording electrodes) were filled with a 2 M NaCl solution saturated with Fast Green FCF, resulting in an impedance of 2-5 MΩ. Mice were anaesthetized with chloral hydrate (400 mg/kg, i.p., using a 2% solution), and placed in a stereotaxic frame equipped with the Stoelting “just for mouse” adaptor. Electrodes were positioned 0.5-1 mm posterior to the interaural line on the midline, and were then lowered into the DRN, usually attained at a depth of 2.5 mm from the brain surface. 5-HT neurons were then encountered over a maximal distance of 1 mm. They were identified using the following criteria: a slow (0.5-2.5 Hz) and regular firing rate and long-duration (0.8-1.2 ms) action potentials, with a positive-negative spike [1]. Spikes were computed by using the Spike 2 software, so that the firing rate was calculated as the mean number of events occurring within a 10 s period. For each neuron, the discharge was monitored during 60 seconds. Each mouse received either spadin (10⁻⁵ M in a 100 μl bolus) or its vehicle (saline). Starting 30 min after the injection, 3 to 4 successive descents were performed along the DRN, for a total of 4-8 cells recorded per animal. Recordings were performed for a maximal duration of 4 hours post-injection.

### Measurement of Hippocampal Neurogenesis

Twenty-four hours after the injection of BrdU (120 mg per kg of body weight in a 300 μl bolus), mice were euthanized and transcardially perfused with 4% cold paraformaldehyde. Serial sections of the brains were cut (40 μm) throughout the entire hippocampus on a vibratome (Leica). Every sixth section throughout the hippocampus was processed for BrdU or doublecortin (DCX) immunohistochemistry, as previously described [1]. For each immunodetection, slides were first incubated overnight at 4° C. with a mouse monoclonal anti-BrdU antibody (1:200; Becton-Dickinson) or a goat anti-DCX (1:400, Santa Cruz Laboratories). For chromogenic immunodetection, sections were then incubated for 1 hour in biotin-conjugated species-specific secondary antibodies (1:100; Vector Laboratories) followed by a peroxidase-avidin complex solution according to the manufacturer's protocol. The peroxidase activity of immune complexes was visualized with DAB staining using the VectaStain ABC kit (Vector Laboratories). For fluorescent double-labeling, performed to determine the cell phenotype, sections were incubated overnight at 4° C. with anti-sheep BrdU (1:200, Interchim), anti-goat DCX (1:200, Santa Cruz Laboratories), or an anti-GFAP (Glial Fibrillary acidic protein, marker for astrocytes, 1:250, Dako). Antibodies were revealed with anti-IgG Alexa 488 or 594-coupled antibodies (1:400; Molecular Probes). All BrdU-labeled cells in the granular cell layer and subgranular zone (SGZ) were counted in each section (n=10 and 5 mice per group) at 400× and 1000× magnification under a light microscope (Olympus) by an experimenter blinded to the study code. The total number of BrdU-positive cells per section was multiplied by 6 to obtain the total number of cells per dentate gyrus. The counting of BrdU/DCX labeled cells was performed using a Laser Scanning Confocal Microscope (TCS SP, Leica) equipped with a DMIRBE inverted microscope.

### Assessement of CREB Activation

Total CREB and pCREB Western Blotting

Mouse brains were dissected on ice. Isolated hippocampi (1-2 mg wet tissue/100 μl) were homogenized in a solubilization buffer containing 20 mM HEPES (pH:7.9), 0.4 M NaCl, 20% (v/v) glycerol, 1% (v/v) Nonidet P-40, 5 mM MgCl₂, 0.5 mM EDTA, 0.1 mM EGTA and protease inhibitor cocktail using a dounce homogenizer. The homogenates were centrifuged 30 min at 15,000×g at 4° C. Supernatants were stored at −70° C. until further use. Protein concentrations were measured using conventional Bradford's method. Samples (50 mg proteins from each experimental group) were resuspended in SDS buffer, sonicated, boiled for 5 min before loading on 10% SDS PAGE gels and electrophoresed for 2 hours at 60 mA. Proteins were then transferred from gels onto Hybond-PVDF membranes (Amersham Biosciences) in blotting buffer (156 mM Tris, 1 M glycine, PBS) for 90 min at 80 mA and blocked with 5% skim milk (Regilait) in PBS for 2 hours at room temperature. Membranes were incubated with the monoclonal anti-pCREB antibody (1:1000, Cell Signaling Technology) overnight at 4° C. Total CREB or tubulin contents were determined after stripping by using 1/1000 dilution of anti-CREB antibody (Cell Signaling Technology) and 1/1000 dilution of anti-tubulin antibody (Sigma-Aldrich). After washing in 0.1% Tween/PBS (four times, 15 min each), secondary anti-mouse or anti-rabbit horseradish peroxidase-conjugated antibody (Amersham Biosciences, 1/10000) was used for 1 h at room temperature. Detection of blotted proteins was performed using ECL plus Western blotting detection reagents (Amersham Biosciences, Orsay, France) with a Las-3000 imaging system (Fujifilm). Relative intensities of the labeled bands were analyzed by densitometric scanning using Image) imaging system. CREB activation was expressed as the ratio between pCREB and total CREB present in each sample.

pCREB Immunostaining in Hippocampal Sections

Free floating sections were permeabilized in 0.1% Triton/PBS for 10 min and blocked with 3% goat serum/PBS for 2 hours at room temperature followed by a single wash in PBS. Sections were then incubated with the monoclonal anti-pCREB antibody (1:300, Cell Signaling Technology) overnight at 4° C. After three washes in PBS, sections were then incubated in biotinylated horse anti-rabbit IgG (Jackson ImmunoResearch, diluted 1:15000) for 2 hours at room temperature. pCREB expression was visualized by 3.3′-diaminobenzidine (DAB) staining using the VectaStain ABC kit (Biovalley). All sections were washed and mounted with Entellan. For BrdU/pCREB immunofluorescence staining, free floating sections of mice injected with BrdU were incubated with the primary antibodies, anti-rabbit pCREB (1:500, Cell Signaling Technology) and anti-mouse BrdU (1:40, Becton-Dickinson). After three washesin PBS, sections were then incubated in anti-IgGAlexa 488- or 594-coupled antibodies (Molecular Probes) for 1 h at room temperature. After drying sections in the dark boxes for 2 hours, they were mounted with Vectashield (Vector laboratories). Confocal microscopy observations were performed with the Laser Scanning Confocal Microscope (TCS SP, Leica) equipped with a DMIRBE inverted microscope, using a Plan Apo 63x/1,4 NA oil immersion objective.

### Corticosterone Assay

Mice were individually housed for at least 12 hours before blood sample collection. Serum samples, collected in the morning were obtained by retroorbital puncture before and 30 min after the end of a 10-min tube restraint. Levels of corticosterone were measured by radioimmunoassay using a commercially available kit (MP Biomedicals).

### Generation of TREK-1 Deficient Mice [53]

TREK-1 genomic clones were isolated from a 129 mouse genomic library by using a TREK-1 cDNA probe and subcloned into pBluescript SK (Stratagene). The Floxed targeting vector was generated from a 7.5 kb Bgl2/EcoR1 restriction fragment containing exon 1-3 of the KCNK2 gene. The vector was designed to allow CRE-mediated deletion of exon 3 which encodes the TM1 domain of the channel. A first loxp sequence was inserted in the 5′ flanking intron of exon 3. Similarly, the PGK-neomycin resistance cassette (neo) was inserted together with a second loxp sequence in the 3′ flanking intron of exon 3. Both loxp sequences were in the same orientation to allow CRE-mediated simultaneous excision of Exon 3 and neo cassette. A copy of the diphteric toxin gene was subcloned adjacent to the homologous region for negative selection of the ES clone. The targeting vector (50 μg) was linearized prior to electroporation into 129-derived embryonic stem cells. After drug selection (G-418, 350 μg/ml), one positive clone (1/288) was identified by Southern blot and PCR analysis. Five highly chimeric males were generated by injection of the targeted ES cells into C57Bl/6J blastocysts. 8-10 weeks old male kenk2^{+/+} and kenk2^{-/-} used for behavioral experiments were from N11 C57Bl/6J derived ^{+/-}F4 intercross littermates. kcnk2^{+/-} (N11 backcross to C57Bl/6J) were crossed to generate ^{+/+} and ^{-/-} male and female littermates (N11F4). Male and female of the same genotype (either ^{+/+} or ^{-/-}) were then crossed to each other to generate large number of animals used for behavioral experiments. For pharmacological studies, we used C57Bl/6J mice as wild-type controls. Pilot studies have shown that the results arising from behavioral experiments performed on littermate's N11F4 kcnk2^{+/+} and C57Bl/6J mice were comparable (data not shown). It is the reason why we considered C57Bl/6J mice as wild-type mice.

### Rat Extracellular Unitary Recordings of DRN 5-HT Neurons.

Single-barreled glass micropipettes (recording electrodes) were filled with a 2 M NaCl solution saturated with Fast Green FCF, resulting in an impedance of 2-5 MΩ. Rats were anaesthetized with chloral hydrate (400 mg/kg, i.p., using a 8% solution), and placed in a stereotaxic frame. A burr hole was drilled on the midline 1 mm anterior to lambda. DRN 5-HT neurons were encountered over a distance of 1 mm starting immediately below the ventral border of the Sylvius aqueduct. They were identified using the following criteria: a slow (0.5-2.5 Hz) and regular firing rate and long-duration (0.8-1.2 ms) action potentials, with a positive-negative spike [46]. Spikes were computed by using the Spike 2 software, so that the firing rate was calculated as the mean number of events occurring within a 10 s period. For each neuron, the discharge was monitored during 60 seconds. Each rat received either spadin (10⁻⁵M in a 500 μl bolus) or its vehicle. Starting 30 min after the injection, 2 to 3 successive descents were performed along the DRN, for a total of 8-12 cells recorded per animal. Recordings were performed for a maximal duration of 4 hours post-injection.

### Behavioral Tests

The behavioral experiments were performed blind to experimental groups and genotype in a quiet room by the same investigator for a given test. All mice were naïve in each behavioral test used.

### Depression Behavioral Tests

Porsolt Forced Swim Test (FST) [22]

Mice (Janvier, France) were placed individually in cylinders (height: 30 cm, diameter: 15 cm) filled with 12 cm deep water (temperature: 22±1° C.) for 6 min. The total period of immobility was recorded during the last 4 min.

Male Sprague-Dawley rats (Janvier, France), weighing 250-300 g were also used for the FST. Rats experienced a pretest session followed 24 h later by a test session. For both the pretest and the test sessions, conducted under low illumination (15 W), the animals were placed in a plastic cylindrical tank (50 cm high by 20 cm in diameter) filled with 40 cm deep water at 24° C. The pretest was carried out for 15 minutes and the test for 5 minutes in the same tank. The total period of immobility was analyzed during the last 4 min.

Tail Suspension Test (TST) [23]

Mice were suspended by the tail. After an “agitation” or “escape-like” behavior, mice adopted an immobile posture, suggested to mirror a state of depression. The immobility time was recorded during a 6 min test session.

Conditioned Suppression of Motility (CSM) Test [25]

Mice were placed in a rectangular cage (20×20×27 cm) with a metallic grid floor, located in a sound-proof room illuminated by a 75 W bulb. There were 12 squares drawn on the floor for counting animal displacements. On the first day, the mouse was left in the test cage for 6 min and received 30 electric footshocks of 1.8 mA during 200 ms (one shock every 12 s.). On the second day, the mouse was again placed in the same cage without receiving electric footshocks (conditioned suppression group). Motility changes were observed by counting the number of squares crossed plus the number of climbing in 6 min. Each control group was treated exactly by the same way, except for the absence of the electric foot-shock. In this test, the mice learned to associate a conditioned stimulus (CS: test chamber) with an unconditioned stimulus (US: footshocks). After the pairing of CS and US, a robust associative memory of the CS-US was formed such that the CS alone could elicit a fear response (behavioral immobility or freezing).

Learned Helplessness (LH) Test [26]

LH training was carried out in 2-way shuttle plexiglas boxes (Imetronic). Each box was divided in 2 equal compartments (19.5×10×13 cm) separated by a wall equipped with, a gate (4.7×4.7 cm) that was closed during LH training but opened automatically during shuttle escape testing. LH was induced in shocked group by administering 360 inescapable, 2-s duration footshocks (0.3 mA intensity), administered once every 10 s over a 1-hour session. Scrambled shocks were delivered by a source shock to a grid floor, which was made of stainless steel bars 2 mm in diameter, spaced 4 mm apart. A non-shocked control group was exposed to the apparatus for an equivalent period of time but did not received shock. 24 hours later, escape testing was performed in the same box, but the animals had the possibility to avoid shocks by moving through the gate to the other side of the box. During testing sessions, all mice were given 30 shuttle escape trials with 30 s intervals between the start of each trial. While on the first five trials, the gate opened at the same time as the shock (0.3 mA), for the 25 remaining trials, the gate opened 2 s after shock onset. Each trial (with a maximal 24 s) duration was terminated when the mouse went into the adjacent compartment. Escape latencies were averaged over 5 trials. Overall escape latencies were computed by averaging escape latencies over 30 trials.

Novelty-Suppressed Feeding Test (NSF) [31]

NSF paradigm is a conflict test that elicits competing motivations: the drive to eat and the fear of venturing into the center of a bright arena. Latency to begin eating is used as an index of anxiety- and depression-like behavior. After 4 day treatment (saline or spadin, i.v.) and 24 hours food-deprivation, mice were presented with a food pellet placed in the center of a brightly plastic box (50×50×20 cm). Latency to begin eating was measured as previously described [31, 1].

Anxiety Behavioral Tests

Elevated plus-maze [54]. The apparatus consisted of two open and two enclosed arms of the same size (45×5 cm) with walls 15 cm high. The arms, constructed of black acrylic, radiate from a common central platform (5×5 cm) to form a plus sign. Open arms were arranged opposite to one another, as were closed arms. The maze was elevated to a height of 45 cm above floor level. Each mouse was placed in the central platform facing one of the open arms. The number of entries into the open and closed arms and the time spent on the open and closed arms were recorded during a 5 min test period. Time spent in the unprotected open arms as well as the number of entries in open arms in relation to the total of arm entries are used as experimental indices of anxiety.

Light/Dark Exploration [55]

The apparatus consisted of a cage (25×40×20 cm) divided into two compartments by a black partition containing a small opening that allows mouse to move from one compartment to the other. One compartment, comprising one-third of the surface area, was made of white plastic and was brightly illuminated. The adjoining smaller compartment was black and dark. Mice were placed in the white compartment and allowed to move freely between the two chambers for 5 minutes. The number of transitions between the two compartments, time spent in the white chamber, and latency to the first transition were recorded. Mice tend to avoid the white compartment. Thus the measures of exploration in this area (time and entrances) were used as experimental indices of anxiety.

Stair Case Test [56]

The apparatus comprised an enclosed staircase consisted of five identical steps (2.5 cm in height×11 cm in width×7.5 cm in length), each made of black plastic. The height of the walls was constant (12.5 cm above the stairs) along the entire length of the staircase. Each mouse was placed individually on the floor of the staircase with its back to the staircase. A step was considered climbed only if the mouse placed all four paws on it. Rearing was recorded when the mouse rose on its hind legs either on the step or against the wall. The number of steps ascended and the number of rearings made in a 3-min period were recorded. Results were expressed as ratio of rearing/step numbers.

### Statistical Analyses.

Data were expressed as mean±S.E.M. Statistical analysis of differences between groups was performed by using unpaired t test or ANOVA (with one or two factors and with or without repeated measured where appropriate). Where F ratios were significant, statistical analyses were extended and post-hoc comparisons made by using LSD, Newman-Keuls or Tukey's test multiple comparison tests. The level of significance was set at P<0.05.

### Results

NTSR3/Sortilin and Spadin Interact with the TREK-1 Channel

In an attempt to detect a physical and functional interaction between the neurotensin receptor and the potassium channel, we first performed an immunoprecipitation of TREK-1 and NTSR3/Sortilin. Experiments were performed on either mouse cortical neurons, or COS-7 cells co-expressing both proteins. Each antibody immunoprecipitated the tested partner, i.e. NTSR3/Sortilin [8] precipitated with the TREK-1 antiserum [16] (FIG. 1A left panel), and TREK-1 with the anti-NTSR3/Sortilin antibody (FIG. 1A right panel), in both COS-7 cells and cortical neurons. We also demonstrated that both endogenous proteins were colocalized in mouse cortical neurons (FIG. 1B). Then, we investigated the influence of NTSR3/Sortilin expression on the sorting of TREK-1 to the plasma membranes. The expression of TREK-1 within the plasma membranes, measured either by preparing purified plasma membranes or by using cell surface biotinylation, was enhanced (by a factor 3 and 6, respectively) when COS-7 cells were cotransfected with NTSR3/Sortilin (FIG. 1C), confirming the interaction between the two proteins, at least during the channel sorting. This interaction between TREK-1 and NTSR3/Sortilin led us to examine whether NT and/or the partial NTSR3/Sortilin propeptide (ie spadin) were able to act on TREK-1 channel activity. We first characterized the affinity of spadin on C13NJ, a microglial cell line expressing only NTSR3/Sortilin as a receptor for NT, and devoid of TREK-1 (not shown). Similarly to NT, spadin bound to NTSR3/Sortilin by displacing the binding of ¹²⁵I-NT with an affinity of 8 nM, identical to that previously found with the full length propeptide [17] (FIG. 1D). Since NT plays a role on C13NJ migration in a wound-healing assay and that the full length propeptide antagonizes this effect [17], we tested in the same assay the spadin effect on NT-induced cell migration. In serum free medium containing 10 nM NT, the number of cells that migrated corresponded to 35.1±2.3% of the number of migrating cells in the presence of 10% fetal calf serum (FCS). In absence of stimulation, only 4±1% of cell migrated. The 10 nM NT-induced cell migration was totally abolished in the presence of 1 μM spadin and remained to the basal level (6.2±1.3%) (Supplementary FIG. 1). This result confirms that spadin displays identical binding and functional properties as those of the full length propeptide. Then, we performed competition experiments between ¹²⁵I-labelled spadin and unlabelled spadin, NT or the N-terminal fragment of the full length propeptide Gln1-Arg16 on membrane homogenates from COS-7 cells transfected or not with TREK-1. FIG. 1E shows that spadin bound specifically to TREK-1 with an affinity of about 10 nM. This binding was selective since NT was unable to displace the binding of ¹²⁵I-spadin and the N-terminal fragment Gln1-Arg16 bound to TREK-1 with a very low affinity (1 μM) close to this reported with NTSR3/sortilin [15] (FIG. 1E). The weak effect of the N-terminal fragment Gln1-Arg16 was confirmed by electrophysiological recordings on TREK-1 transfected COS-7 cells (Supplementary FIG. 2). We also performed association kinetics of ¹²⁵I-labelled spadin on whole COS-7 cells expressing TREK-1 at 37° C. The radioactivity associated with cells reached a plateau within 30 min. Removal of surface-bound radioactivity by acid-NaCl wash revealed that about 80% of total ¹²⁵I-labelled spadin bound at this time was intracellular, indicating that spadin was internalized with TREK-1 following interaction (FIG. 1F). These data strongly suggest that NTSR3/Sortilin constitutes a sorting partner of the TREK-1 channel. We hypothesed that when both proteins reach the plasma membrane, the propeptide, which is cleaved in the Golgi apparatus, can be released. Then, it may bind to NTSR3/Sortilin and/or to TREK-1 for tuning the channel activity, by blocking a part of the expressed channels, and by promoting their internalization. However, for such a mechanism to be functionally effective under in vivo physiological conditions, the propeptide has to be released into the blood circulation. To validate this possibility, we therefore developed the Alpha Screen™ (Amplified Luminescent Proximity Homogenous Assay) technology for dosing the propeptide or spadin in serum samples from mice [18,19,20]. This method is a bead-based non radioactive and homogenous proximity assay used to measure the interaction between biological binding partners (FIG. 2A-B). The principle of this technology relies on the use of a Donor bead and an Acceptor bead that generate a light signal when brought into proximity (<200 nm). Upon laser excitation at 680 nm, the Donor beads, containing a photosensitizer, will generate short-lived singlet oxygen that can diffuse only a short distance before returning to the ground state. The Acceptor beads, containing chemiluminescers and fluorophores, will emit an amplified light signal measurable at 600 nm (FIG. 2A). Using this approach, we calculated seric propeptide concentrations of 5 groups of 6 littermate mice. Interestingly, the mean concentration values of the 5 groups were very close to each other, with a value of about 10 nM (FIG. 2C and Supplementary FIG. 3). These data clearly indicated that the propeptide is released into the blood circulation. On this basis, we next investigated its effects, and by extension those of spadin as well, on TREK-1 channel activity.

### Effects of Spadin on the TREK-1 Channel Activity

As previously described [21], TREK-1 basal channel activity was strongly and reversibly activated by arachidonic acid (aa, 10 μM), which induced a typical TREK-1 background current, characterized by outward rectification reversed at the predicted value for E_{K+}. Using the whole-cell patch-clamp technique on TREK-1 transfected COS cells, we first assessed the ability of the full length peptide to inhibit the TREK-1 channel activity. COS-7 cells were chosen because they weakly express the NTSR3/Sortilin receptor (not shown). Indeed, 500 nM of propeptide was able to block 41±5% (n=4) of the aa stimulated TREK-1 current measured at 0 mV (FIG. 3A). Then, we tested spadin in the same experimental conditions. As expected [15], we found that spadin displayed a better affinity than the propeptide, since 100 nM of spadin were able to block 63±12% (n=16) of the TREK-1 current stimulated by aa (FIG. 3B). A spadin dose-response experiment indicated an IC₅₀ value of 70.7 nM at 0 mV (FIG. 3C).

In order to confirm the action of spadin on TREK-1, by using brain slices we directly recorded hippocampal CA3 pyramidal cells, a cellular network that endogenously expresses both TREK-1 and NTSR3/Sortilin [7,8]. FIG. 3D depicts the currents obtained following a ramp of potential in a CA3 neuron in the presence of a cocktail of K+ blockers that have no effect on TREK-1 [21]. In 12 out of 28 recorded neurons, arachidonic acid increased the amplitude of the remaining current by 23.3+4.8% (n=12). Spadin blocked 90.8±6.0% (n=12) of this effect. Interestingly, the peptide alone inhibited 14.9+5.6% (n=8) of the current recorded in the presence of potassium blockers (not shown), as did fluoxetine (13.0+3.8%, n=5) (data not shown). Even if an effect of spadin on cationic channels cannot be totally excluded, the inhibitory effect of spadin on arachidonic acid-induced current in CA3 neurons from wild-type mice was totally absent in the same experimental conditions in the kcnk2^{-/-} mice (FIG. 3E). This result clearly demonstrates that the current blocked by spadin is supported by TREK-1 channel. The inhibitory effects of spadin on endogenous TREK-1 were also measured in cultured pyramidal neurons from hippocampus (49.7% inhibition with 1 μM of spadin, Supplementary FIG. 4) and in the non neuronal βTC3 pancreatic cell line (36% inhibition with 1 μM of spadin, FIG. 3F) that endogenously express both proteins (Mazella, personal communication).

### Effect of Spadin on the Dorsal Raphe Nucleus (DRN) 5-HT Neurotransmission

Since we have previously demonstrated that the deletion of the TREK-1 gene results in an increase of the 5-HT neuron firing rate in the Dorsal Raphe Nucleus (DRN) [1], we tested the effect of spadin on the same neurons. We performed unitary extracellular recordings of these 5-HT neurons in anesthetized animals. We constituted two groups of mice, which received via an i.p. injection either spadin (10⁻⁵ M in a 100 μl bolus) or its vehicle (distilled water). Starting 30 min after the injection, 3 to 4 successive descents were performed along the DRN, for a total of 4-8 cells recorded per animal (examples are given in FIG. 3G). For each neuron, the discharge was monitored during 60 seconds. In vehicle-injected mice, we found a value of 1.26±0.27 Hz, whereas after administration of spadin, the mean firing rate of DRN 5-HT neurons was significantly elevated up to 3.1±0.7 Hz (FIG. 3H) (one-way ANOVA, F(_{1,36})=4.4, P<0.05), corresponding to a +146% increase. As shown in FIG. 3G (right panel), several neurons found in spadin-injected mice discharged at up to 3.5 or even 6 Hz, whereas most of the frequencies found in the saline group were in a normal range (1-1.7 Hz) (FIG. 3G left panel). The average 5-HT neuron firing activity in spadin-treated animals was almost identical to that observed in kcnk2^{-/-} mice [1] (FIG. 3H). Very similar results were obtained when spadin was i.p. injected in rats (Supplementary FIG. 5A-B).

Taken together, these results indicate that TREK-1 and NTSR3/Sortilin are not only associated within the plasma membrane, but that spadin interactes directly with TREK-1 to functionally inhibit its activity. These results prompted us to test thereafter the antidepressant-like effects of spadin in behavioral, morphological and molecular models.

### Acute, Subchronic and Chronic Spadin Treatments Induce Antidepressant Effects

We used five behavioral tests predicting an antidepressant response (FST, TST, CMST, LH and NSF) similar to these used in our previous work on the depression-resistant phenotype of TREK-1 deficient mice [1]. Spadin efficacy was first assessed in the forced swimming test (FST) [22], which is a highly reliable predictor for antidepressant potential [13]. Spadin was administered 30 min before the test by intracerebroventricular (i.c.v.), intravenous (i.v.) or intraperitoneal (i.p.) route at doses of 10⁻⁴ to 10⁻⁸ M. Its effects were compared to the behavior observed in kcnk2^{-/-} mice and in wild-type mice treated with the efficient SSRI fluoxetine (i.p, 3 mg/kg). When placed in an inescapable cylinder of water, spadin-treated mice exhibited reduced floating or immobility times in the three modes of injection with respect to their saline-treated counterparts (FIG. 4A). The immobility is interpreted as “a state of despair”, in that the animal is believed to have lost its motivation for escape-oriented behaviors. The dose-responses of spadin showed that the highest reduced immobility times (P<0.001) were observed at the dose of 10⁻⁷ M in i.c.v. (66.8%), 10⁻⁶ M in i.v. (62.9%) and 10⁻⁸ M in i.p. (55.30%) administration. The magnitude of the antidepressant behavior was similar to that observed in fluoxetine-treated wild-type and saline-injected kcnk2^{-/-} mice. Then, we determined the effect of an acute i.v. spadin administration (10⁻⁶ M) in the Tail Suspension Test (TST, FIG. 4B), which is often used to predict antidepressant efficacy [23,24], and in the test of Conditioned Suppression of Motility (CMST, FIG. 4C), sensitive to antidepressants but not to anxiolytic drugs [25]. In the TST, injection of spadin in wild-type mice 30 min before the test significantly reduced immobility times when compared to saline-treated wild-type mice (P<0.001). The antidepressant effect was not statistically different to that observed in fluoxetine-injected mice or kcnk2^{-/-} mutants (P>0.05; FIG. 4B). In the CMST, shocked mice treated with saline displayed a marked suppression of motility (CS: conditioned suppression, 9.1% of the saline non-shocked group) when they were returned to the cage in which they had previously received electric shocks (FIG. 4C). Similarly to what was observed in saline-treated kcnk2^{-/-} mice, the administration of spadin (i.v., 10⁻⁶ M) significantly reduced (by 84.4%) the CS of motility without increasing motility in the corresponding non-shocked group (FIG. 4C). In these three tests (FST, TST and CMST), the injection of spadin in kcnk2^{-/-} mice did not induce any change (FIG. 4A-C), indicating that there was no additional effects of spadin in the absence of the TREK-1 channel. The subsequent experiments were therefore performed only in wild-type mice. We subjected mice to the Learned Helplessness test (LH, FIG. 4D-E), validated as a sensitive model of depression [13,26]. Compared with non-shocked mice (i.e. that had not been exposed to inescapable shocks), learned helpless (shocked) mice treated with saline showed a significant increase of escape latencies, when tested for their escape performance abilities one day after exposure to inescapable shocks (FIG. 4D-E). However, an acute spadin treatment (i.v., 10⁻⁶M) provoked significant reduced escape latencies after training (25.4%) compared to saline-treated mice, demonstrating a strong antidepressant effect. Because changes in the motor activity induced by the different drugs used could influence the results, the motor behavior was measured following i.p. treatments. Neither spadin nor fluoxetine had any effect on mouse locomotion analyzed in short- or long-time after the drug injection (Supplementary FIG. 6).

Current antidepressants are clinically effective only after several weeks of administration. They increase the efficacy of 5-HT transmission at the postsynaptic levels [27, 28] but the initial elevation of 5-HT also induces the stimulation of inhibitory 5-HT_{1A} autoreceptors within the DRN, counteracting the facilitation of 5-HT transmission related to terminal reuptake blockade [28]. The existence of this presynaptic effect is believed to be responsible for the 2 to 6 weeks delay before the onset of the antidepressant's therapeutic action, as this period corresponds to the time required for 5-HT_{1A} autoreceptors to desensitize [28]. Based on these observations, it has been proposed that a direct facilitation of 5-HT firing rate in the DRN should be a requirement for a faster onset of antidepressant action [29]. Interestingly, we observed an increase in the firing activity of DRN 5-HT neurons (FIG. 3G). Obviously, such results raise the possibility that spadin could exert a rapid onset of action. Hence, we tested the potential antidepressant effect of spadin administered during 4 days (subchronic treatment) using both the FST and TST tests. As shown in FIG. 5A-B, a 4-day treatment with spadin (i.v., 10⁻⁶M) significantly reduced the time spent immobile by 43.2% in FST (P<0.01) (FIG. 5A) and by 28.1% in TST (FIG. 5B). In contrast, as previously observed [30], subchronic fluoxetine treatment had no effect when compared with saline (P>0.05). Both spadin and fluoxetine administered during 15 days (chronic treatment) significantly reduced the time of immobility to a similar extend (around 30%) in the FST paradigm (FIG. 5 C). This result show that the antidepressant effect of spadin reached a maximal level after 4 days and maintained the same potency following long-term administration (15 days). The Novelty Suppressed Feeding test (NSF) is usually carried out for demonstrating antidepressant efficacy after chronic, but not acute treatment [31]. Mice treated with spadin (i.v., 10⁻⁶ M) for 4 days showed a significant decrease in latency to feed relative to saline-injected animals (FIG. 5D). As previously described [30], a 4-day regimen with fluoxetine (i.p., 3 mg/kg) had no effect in the same condition (FIG. 5D). None of the drugs tested produced a significant change in food consumption when mice returned in their home cage immediately after the test (data not shown).

To verify that the antidepressant effects of spadin were not species-specific, we also tested its efficiency in rats, by using the FST test. An acute spadin injection (i.p., 10⁻⁵ M) 30 min before the test significantly reduced immobility time when compared with saline-treated rats (P<0.001). The antidepressant effect was not statistically different to that obtained with fluoxetine (20 mg/kg)-injected mice or kcnk2^{-/-} mice (P>0.05; Supplementary FIG. 5C).

Acute Spadin Treatment does not Affect Anxiety-Related Behaviors

Stress and anxiety disorders lead to profound suffering and disability, which contributes to the development of depression in humans and play a role in the severity and the recurrence of the disease [32]. The connection between stress, anxiety and depression is often associated with elevated cortisol levels in depressed patients [33]. We have previously demonstrated that the deletion of the TREK-1 gene results in a hypoactivity of the hypothalamic-pituitary-adrenal (HPA) axis, known to be involved in the control of stress [1]. Here, we tested whether spadin (i.p. 10⁻⁵ M) reduced corticosterone levels 30 min after a 10 min-tube restraint, a paradigm known to activate the HPA axis [34]. FIG. 6A shows that the increase in corticosterone levels induced by stress were reduced by 79.5 and 59.1% in spadin- and fluoxetine-treated mice with respect to saline-treated animals.

Thereafter, to study whether spadin affects anxiety-related behaviors, we investigated its anxiolytic profile in the three mouse models of anxiety (elevated plus-maze, light-dark exploration, staircase) that are the most commonly used [35]. Their most important feature resides in the predictive validity to detect anxiolytic potential. Avoidance behaviours are reduced by treatments with clinically efficacious anxiolytics, mainly by the benzodiazepine agonist class, including diazepam [36]. In the elevated plus-maze test, compared to diazepam (i.p., 0.5 mg/kg), which significantly reduced the time spent into the aversive open arms of the test apparatus (*P<0.05), spadin (i.p., 10⁻⁵M) had no effect (P>0.05) versus saline-treated mice (FIG. 6B). Similarly, in the light/dark exploration test, spadin-treated mice did not make more transitions from the dark to the light compartment than did mice treated with saline (P>0.05). In contrast, diazepam significantly induced an increase in the number of light/dark transitions (*P<0.05) (FIG. 6C). The staircase paradigm combines step-climbing, which serves as an index of exploratory and locomotor activity, and rearing, which serves as an indicator of anxiety. Exposure to diazepam induced a significant reduction in both rearing and step ascending behaviours, leading to a decrease of the rearing/step numbers ratio (**P<0.01, FIG. 6D). In contrast, spadin had no effect as compared to saline-treated mice (P>0.05). Together, these results demonstrate that spadin has no anxiolytic activity, when compared with the well-known diazepam.

Since spadin exerted efficient effects after i.p. or i.v. administration, we evaluated its ability to pass through the blood-brain barrier. ¹²⁵I-labelled spadin (1 nmol of spadin plus 2×10⁶ cpm ¹²⁵I-spadin) was i.v. injected and animals were sacrificed 30 min following injection. The brains were rapidly removed and homogenized. The radioactivity recovered in the brain was acid-extracted, quantified and analyzed by HPLC. These experiments indicated that 1/1000 of spadin was recovered in the brain after i.v. injection (data not shown). Identical amounts of spadin were recovered in the brain after i.p. injection under the same experimental conditions. The concentration of spadin recovered into the brain was estimated to stand around 10 nM, a concentration that corresponds to the affinity of spadin for TREK-1 and sufficient to be active on TREK-1 channel activity. This value was also in the same range of the IC₅₀ determined by electrophysiological measurements (FIG. 3C).

Effects of a 4-Day Treatment with Spadin on CREB-Phosphorylation and Hippocampal Neurogenesis

SSRIs and tricyclics are known to enhance neurogenesis in the subgranular zone (SGZ) of the dentate gyrus, but only after 2 or 3 weeks of a chronic treatment [31,37]. The concomitant increases of both the transcription factor cAMP response element-binding protein (CREB) and hippocampal neurogenesis in response to chronic antidepressant treatment, but not to non-antidepressant psychotic drugs, strongly suggest that CREB regulates hippocampal neurogenesis [13,38]. We tested therefore whether spadin was able to induce an increase in hippocampal neurogenesis and a faster activation of CREB. We analyzed the neurogenesis in the dentate gyrus of the mouse hippocampus, by counting the number of progenitor cells that incorporate the DNA synthesis marker 5-bromo-2′ deoxyuridine (BrdU) and that are differentiated into mature neurons [31,37]. Interestingly, in the SGZ, a 4-day treatment with spadin (i.p. 10⁻⁵M) significantly increased by two-fold the number of BrdU-positive cells with respect to saline conditions (FIG. 7A-B). The neurogenic effect of spadin was maintained after a long-term administration (15 days, FIG. 7C). In contrast, a 4-day regimen with fluoxetine had no effect on neurogenesis, but fluoxetine induced a significant increase in the number of BrdU-positive cells when it was administered during 15 days (FIG. 7C). Dual labeling of BrdU and doublecortin (DCX), a specific marker of neuronal precursors [39] revealed that 85.2% of BrdU-labelled cells expressed DCX (FIG. 7A right bottom panel, 7B). No colocalization of BrdU-positive cells with the astroglial marker GFAP (glial fibrillary acidic protein) was observed (FIG. 7A left bottom panel).

The next step was to determine whether the enhanced adult spadin-induced neurogenesis was related to an increased hippocampal activation of CREB, as measured by its phosphorylation into pCREB. Compared with saline-treated mice, a 4-day treatment with spadin (i.p., 10⁻⁵M) induced a large increase of pCREB labeling, restricted to the specific SGZ region of mouse hippocampal tissue sections (FIG. 7D). The counting of pCREB⁺ cells revealed that the spadin administration during 4 days significantly led to a two-fold increase in the number of pCREB-labelled neurons when compared with the saline group (P<0.001, FIG. 7E). Western blot analysis, which showed an immunoreactive band at 46 kDA corresponding to the phosphorylated active form of CREB, confirmed that a 4-day treatment with spadin (i.p., 10⁻⁵ M) stimulated the hippocampal phosphorylation of CREB, whereas the amount of total CREB remained unchanged (FIG. 7F). Quantification of blots indicated a significant 4-fold stimulation of pCREB within the hippocampus extracts. To examine the relationship between pCREB and neurogenesis, expression of pCREB in newborn cells, visualized by DXC labeling was examined by immunofluorescence. Double labeling for pCREB and DCX demonstrated a colocalization of pCREB expression in several precursor neurons in the presence of spadin (FIG. 7G). These data pointed out that spadin induced a specific and rapid onset of neurogenesis and CREB activation in adult brain mice.

### REFERENCES

1. Heurteaux C, Lucas G, Guy N, El Yacoubi M, Thümmler S, et al. (2006) Deletion of TREK-1, a background potassium channel, results in a depression-resistant phenotype. Nature Neurosci 9: 1134-1141.
2. Chemin J, Girard C, Duprat F, Lesage F, Romey G, et al. (2003) Mechanisms underlying excitatory effects of group I metabotropic glutamate receptors via inhibition of 2P domain K+ channels. EMBO J 22: 5403-5411.
3. Lesage F, Lazdunski M (2000) Molecular and functional properties of two pore domain potassium channels. Am J Physiol 279: 793-801.
4. Perlis R H, Moorjani P, Fagerness J, Purcell S, Trivedi M H, et al. (2008) Pharmacogenetic Analysis of Genes Implicated in Rodent Models of Antidepressant Response Association of TREK-1 and Treatment Resistance in the STAR(*)D Study. Neuropsychopharmacology 33: 2810-2819.
5. Sandoz G, Thummler S, Duprat F, Feliciangeli S, Vinh J, et al. (2006) AKAP150, a switch to convert mechano-, pH- and arachidonic acid-sensitive TREK K(+) channels into open leak channels. EMBO J 25: 5864-5872.
6. Sandoz G, Tardy M P, Thummler S, Feliciangeli S, Lazdunski M, et al. (2008) Mtap2 is a constituent of the protein network that regulates twik-related K+ channel expression and trafficking. J Neurosci 28: 8545-8552.
7. Hervieu G J, Cluderay J E, Gray C W, Green P J, Ranson J L, et al. (2001) Distribution and expression of TREK-1, a two-pore-domain potassium channel, in the adult rat CNS. Neuroscience 103: 899-919.
8. Sarret P, Krzywkowski P, Segal L, Nielsen M S, Petersen C M, et al. (2003) Distribution of NTS3 receptor/sortilin mRNA and protein in the rat central nervous system. J Comp Neurol 461: 483-505.
9. Mazella J, Zsurger N, Navarro V, Chabry J, Kaghad M, et al. (1998) The 100-kDa neurotensin receptor is gp95/sortilin, a non-G-protein-coupled receptor. J Biol Chem 273: 26273-26276.
10. Nielsen M S, Madsen P, Christensen E I, Nykjaer A, Gliemann J, et al. (2001) The sortilin cytoplasmic tail conveys Golgi-endosome transport and binds the VHS domain of the GGA2 sorting protein. Embo J 20: 2180-2190.
11. Lefrancois S, Zeng J, Hassan A J, Canuel M, Morales C R (2003) The lysosomal trafficking of sphingolipid activator proteins (SAPs) is mediated by sortilin. Embo J 22: 6430-6437.
12. Nykjaer A, Lee R, Teng K K, Jansen P, Madsen P, et al. (2004) Sortilin is essential for proNGF-induced neuronal cell death. Nature 427: 843-848.
13, Krishnan V, Nestler E J (2008) The molecular neurobiology of depression. Nature 455: 894-902.
14. Munck Petersen C, Nielsen M S, Jacobsen C, Tauris J, Jacobsen L, et al. (1999) Propeptide cleavage conditions sortilin/neurotensin receptor-3 for ligand binding. Embo J 18: 595-604.
15. Westergaard U B, Sorensen E S, Hermey G, Nielsen M S, Nykjaer A, et al. (2004) Functional organization of the sortilin Vps10p domain. J Biol Chem 279: 50221-50229.
16. Maingret F, Lauritzen I, Patel A J, Heurteaux C, Reyes R, et al. (2000) TREK-1 is a heat-activated background K(⁺) channel. EMBO J 19: 2483-2491.
17. Martin S, Vincent J P, Mazella J (2003) Involvement of the neurotensin receptor-3 in the neurotensin-induced migration of human microglia. J Neurosci 23: 1198-1205.
18. Taouji S, Dahan S, Bossé R, Eric C (2009) Current Screen Based on the AlphaScreen Technology for Deciphering Cell Signaling Pathways. Current Genomics 10: 93-101.
19. Mills N L, Shelat A A, Guy R K (2007) Assay Optimization and Screening of RNA-Protein Interactions by AlphaScreen. J Biomol Screen 12: 946-955.
20. Caruso M E, Jenna S, Beaulne S, Lee E H, Bergeron A, et al. (2005) Biochemical clustering of monomeric GTPases of the Ras superfamily. Mol Cell Proteomics 4: 936-944.
21. Patel A J, Honore E, Maingret F, Lesage F, Fink M, et al. (1998) A mammalian two pore domain mechano-gated S-like K+ channel. EMBO J 17: 4283-4290.
22. Porsolt R D, Le Pichon M, Jalfre M (1977) Depression: a new animal model sensitive to antidepressant treatments. Nature 266: 730-732.
23. Steru L, Chemat R, Thierry B, Simon P (1985) The tail suspension test: a new method for screening antidepressants in mice. Psychopharmacology (Berl) 85: 367-370.
24. Nestler E J, Barrot M, DiLeone R J, Eisch A J, Gold S J, et al. (2002) Neurobiology of depression. Neuron 34: 13-25.
25. Kameyama T, Nagasaka M, Yamada K (1985) Effects of antidepressant drugs on a quickly-learned conditioned-suppression response in mice. Neuropharmacology 24: 285-290.
26. Caldarone B J, George T P, Zachariou V, Picciotto M R (2000) Gender differences in learned helplessness behavior are influenced by genetic background. Pharmacol Biochem Behav 66: 811-817.
27. Haddjeri N, Blier P, de Montigny C (1998) Long-term antidepressant treatments result in a tonic activation of forebrain 5-HT1A receptors. J Neurosci 18: 10150-10156.
28. Blier P, de Montigny C (1999) Serotonin and drug-induced therapeutic responses in major depression, obsessive-compulsive and panic disorders. Neuropsychopharmacology 21: 91S-98S.
29. Blier P (2001) Possible neurobiological mechanisms underlying faster onset of antidepressant action. J Clin Psychiatry 62 Suppl 4: 7-11; discussion 37-40.
30. Cryan J F, Page M E, Lucki I (2005) Differential behavioral effects of the antidepressants reboxetine, fluoxetine, and moclobemide in a modified forced swim test following chronic treatment. Psychopharmacology (Berl) 182: 335-344.
31. Santarelli L, Saxe M, Gross C, Surget A, Battaglia F, et al. (2003) Requirement of hippocampal neurogenesis for the behavioral effects of antidepressants. Science 301: 805-809.
32. Sheline Y I, Wang P W, Gado M H, Csernansky J G, Vannier M W (1996) Hippocampal atrophy in recurrent major depression. Proc Natl Acad Sci USA 93: 3908-3913.
33. Holsboer F (2001) Stress, hypercortisolism and corticosteroid receptors in depression: implications for therapy. J Affect Disord 62: 77-91.
34. Hermann G, Beck F M, Tovar C A, Malarkey W B, Allen C, et al. (1994) Stress-induced changes attributable to the sympathetic nervous system during experimental influenza viral infection in DBA/2 inbred mouse strain. J Neuroimmunol 53: 173-180.
35. Cryan J F, Holmes A (2005) The ascent of mouse: advances in modelling human depression and anxiety. Nat Rev Drug Discov 4: 775-790.
36. Rodgers R J (1997) Animal models of ‘anxiety’: where next? Behav Pharmacol 8: 477-496; discussion 497-504.
37. Malberg J E, Schechter L E (2005) Increasing hippocampal neurogenesis: a novel mechanism for antidepressant drugs. Curr Pharm Des 11: 145-155.
38. Carlezon W A, Jr., Duman R S, Nestler E J (2005) The many faces of CREB. Trends Neurosci 28: 436-445.
39. Couillard-Despres S, Winner B, Schaubeck S, Aigner R, Vroemen M, et al. (2005) Doublecortin expression levels in adult brain reflect neurogenesis. Eur J Neurosci 21: 1-14.
40. Gordon J A, Hen R (2006) TREKing toward new antidepressants. Nat Neurosci 9: 1081-1083.
41. Kessler R C, Berglund P, Demler O, Jin R, Merikangas K R, et al. (2005) Lifetime prevalence and age-of-onset distributions of DSM-IV disorders in the National Comorbidity Survey Replication. Arch Gen Psychiatry 62: 593-602.
42. Hermey G (2009) The Vps10p-domain receptor family. Cell Mol Life Sci 66: 2677-2689.
43. Chen Z Y, Ieraci A, Teng H, Dall H, Meng C X, et al. (2005) Sortilin controls intracellular sorting of brain-derived neurotrophic factor to the regulated secretory pathway. J Neurosci 25: 6156-6166.
44. Altar C A (1999) Neurotrophins and depression. Trends Pharmacol Sci 20: 59-61.
45. Nibuya M, Nestler E J, Duman R S (1996) Chronic antidepressant administration increases the expression of cAMP response element binding protein (CREB) in rat hippocampus. J Neurosci 16: 2365-2372.
46. Lucas G, Debonnel G (2002) 5-HT4 receptors exert a frequency-related facilitatory control on dorsal raphe nucleus 5-HT neuronal activity. Eur J Neurosci 16: 817-822.
47. Lucas G (2008) Fast-acting antidepressants: are we nearly there? Expert Rev Neurother 8: 1-3.
48. Thome J, Sakai N, Shin K, Steffen C, Zhang Y J, et al. (2000) cAMP response element-mediated gene transcription is upregulated by chronic antidepressant treatment. J Neurosci 20: 4030-4036.
49. Nibuya M, Morinobu S, Duman R S (1995) Regulation of BDNF and trkB mRNA in rat brain by chronic electroconvulsive seizure and antidepressant drug treatments. J Neurosci 15: 7539-7547.
50. Clancy B M, Czech M P (1990) Hexose transport stimulation and membrane redistribution of glucose transporter isoforms in response to cholera toxin, dibutyryl cyclic AMP, and insulin in 3T3-L1 adipocytes. J Biol Chem 265: 12434-12443.
51. Sadoul J L, Mazella J, Amar S, Kitabgi P, Vincent J P (1984) Preparation of neurotensin selectively iodinated on the tyrosine 3 residue. Biological activity and binding properties on mammalian neurotensin receptors. Biochem Biophys Res Commun 120: 812-819.
52. Duprat F, Lesage F, Fink M, Reyes R, Heurteaux C, et al. (1997) TASK, a human background K+ channel to sense external pH variations near physiological pH. Embo J 16: 5464-5471.
53. Heurteaux, C., et al. (2004) TREK-1, a K(+) channel involved in neuroprotection and general anesthesia. Embo J 23: 2684-95.
54. Gross, C., et al. (2002) Serotonin1A receptor acts during development to establish normal anxiety-like behaviour in the adult. Nature 416: 396-400.
55. Welch, J. M., et al. (2007) Cortico-striatal synaptic defects and OCD-like behaviours in Sapap3-mutant mice. Nature 448: 894-900.
56. Cryan, J. F. et al. (2003) Antidepressant and anxiolytic-like effects in mice lacking the group III metabotropic glutamate receptor mGluR7. Eur J Neurosci 117: 2409-2417.
57. Mazella J. et al. (2010) A sortilin-derived peptide, targeting rodent TREK-1 channels: a new concept in the antidepressant drug design. PLoS Biol. 2010 Apr. 13; 8(4):e1000355.
58. International Application WO 2009/103898.
59. Wong, M. & Licinio (2001) Research and treatment approaches to depression. Nat Rev Neurosci 2: 343-351.
60. Moller H J. (2003) Suicide, suicidality and suicide prevention in affective disorders. Acta Psychiatr Scand 418(suppl): 73-80.

All of the references listed above and cited in the specification are hereby incorporated by reference in their entirety.

1. A method of diagnosing depression in a subject, comprising analyzing a biological sample from a subject in need of diagnosis of depression for the expression of spadin, detecting and measuring the amount of spadin in the sample, and diagnosing depression in the subject if an increase in spadin expression in the biological sample is detected compared to a control.

2. A method of diagnosing depression in a subject, comprising analyzing a biological sample from a subject in need of diagnosis of depression for the expression of spadin, detecting and measuring the amount of spadin in the sample, and diagnosing depression in the subject if a decrease in spadin expression in the biological sample is detected compared to a control.

3. The method of claim 1, wherein the biological sample is a body fluid selected from the group consisting of blood, blood plasma, serum, bone marrow, stool, synovial fluid, lymphatic fluid, cerebrospinal fluid, sputum, urine, mother's milk, sperm, exudates and mixtures thereof.

4. The method of claim 1, wherein the control is the amount of spadin in a sample from a subject without depression.

5. The method of claim for 1, wherein the control is the amount of spadin in a sample from a subject with depression.

6. The method of claims 1, wherein analyzing spadin expression comprises using a probe.

7. The method of claim 6, wherein the probe is an antibody that specifically binds spadin.

8. The method of claim 7, wherein the antibody binds SEQ ID NO: 1.

9. The method of claim 6, wherein the probe is a nucleic acid probe that binds spadin-encoding nucleic acid.

10. The method of claim 1, wherein analyzing the biological sample for spadin expression comprises use of nucleic acid amplification.

11. The method of claim 1, which is carried out in combination with other methods of diagnosing depression.

12. A method of monitoring subjects being treated for depression, comprising: a) detecting and measuring the amount of spadin in a biological sample from a subject being treated for depression and b) comparing the amount in a) to the amount of spadin in a control, wherein a change in the amount of spadin is an indication that the treatment is effective.

13. A method of monitoring depressive subjects in remission, comprising: a) detecting and measuring the amount of spadin in a biological sample from a subject thought to be in remission for depression and b) comparing the amount in a) to the amount of spadin in a healthy control, wherein when the amount in a) is similar to the amount in the healthy control, there is an indication that the subject is in remission.

14. A method of determining the course of depression in a subject comprising: a) measuring at a first time, the amount of spadin in a biological sample from the subject; b) measuring, at a second time, the amount of spadin in a biological sample from the subject; and c) comparing the first measurement with the second measurement; wherein the comparative measurements determine the course of depression in the subject.

15. A method of identifying a candidate compound for treating depression, the method comprising determining whether the candidate compound inhibits currents mediated by the TREK-1 channel, wherein when the candidate compound inhibits currents mediated by the TREK-1 channel, it is a candidate compound for treating depression.

16. A kit comprising a detectably labeled probe for detecting spadin in a biological sample.

17. The method of claim 2, wherein the biological sample is a body fluid selected from the group consisting of blood, blood plasma, serum, bone marrow, stool, synovial fluid, lymphatic fluid, cerebrospinal fluid, sputum, urine, mother's milk, sperm, exudates and mixtures thereof.

18. The method of claim 2, wherein the control is the amount of spadin in a sample from a subject without depression.

19. The method of claim for 2, wherein the control is the amount of spadin in a sample from a subject with depression.

20. The method of claims 2, wherein analyzing spadin expression comprises using a probe.

21. The method of claim 20, wherein the probe is an antibody that specifically binds spadin.

22. The method of claim 21, wherein the antibody binds SEQ ID NO: 1.

23. The method of claim 20, wherein the probe is a nucleic acid probe that binds spadin-encoding nucleic acid.

24. The method of claim 2, wherein analyzing the biological sample for spadin expression comprises use of nucleic acid amplification.

25. The method of claim 2, which is carried out in combination with other methods of diagnosing depression.
